(19) United States
(12) Patent Application Publication
(10) Pub. No.: US 2023/0397856 A1
(43) Pub. Date: Dec. 14, 2023

(54) AUTOMATION COMPATIBLE COLLECTION DEVICE FOR BIOLOGICAL FLUIDS

(71) Applicant: Rhinostics Inc., Waltham, MA (US)

(72) Inventors: Cheri Walker, Wayland, MA (US); Richard Novak, Waltham, MA (US); Anna Simpson, Waltham, MA (US); Christopher Negus, Waltham, MA (US)

(21) Appl. No.: 18/330,087

(22) Filed: Jun. 6, 2023

Related U.S. Application Data
(60) Provisional application No. 63/349,397, filed on Jun. 6, 2022.

(51) Int. Cl. A61B 5/15; B25J 15/08

(52) U.S. Cl. CPC A61B 5/150351; A61B 5/150099; A61B 5/150824; A61B 5/150358; A61B 5/150755; B25J 15/08; A61B 2010/0216

(57) ABSTRACT

This application relates to an automation compatible collection device for use in collecting samples, e.g., biological fluids and other samples. In some embodiments, the collection device can include a capillary for collecting samples from a biological location of a body and a cap configured to interface with an automation device.

### CROSS-REFERENCE TO RELATED APPLICATIONS

This application claims priority to and the benefit of U.S. provisional patent application Ser. No. 63/349,397, filed on Jun. 6, 2022, the disclosure of which is herein incorporated by reference in its entirety.

### FIELD OF THE INVENTION

This invention generally relates to collection devices used for collection of samples and, more particularly, to automation compatible collection devices having features adapted to collect biological fluids and interact with automated equipment and related methods.

### BACKGROUND

Capillary blood collection has been used for decades to collect biological samples from patients. Capillary blood can be obtained by pricking a skin surface of the patient to cause a volume of blood to appear on the surface. The specimen can then be collected with a pipette, cuvette, dried blood card, microfluidic device, or capillary device. In many instances, the sample is then placed on a glass slide or a piece of filter paper for storage and/or further testing. In other instances, the sample is transported back to a laboratory for testing or is used directly on a point of care device at the location of sample collection.

Blood collection continues to occur in a physician's office but is increasingly being moved to self-collection by the patient in the home setting and then transported back to a centralized laboratory for testing. It is neither cost-effective nor practical to have a phlebotomist in the medical office to perform blood collection. Sample collection by the physician or in the home by the patient can be challenging and transport back to the centralized laboratory at ambient temperature is an important requirement necessary to enable broader blood testing menus using capillary blood. Finally, plasma separations at the patient site currently employs devices that are expensive and have challenges when they are brought into the testing laboratory.

When the whole blood and serum samples arrive in the laboratory, transferring the into the diagnostic workflow is challenging, laborious, prone to human errors and contamination, and costly. Additionally, the performance and quality of dried blood cards, microfluidic devices, capillary collection devices varies greatly among commercially available collection devices. Further, there is not always compatibility between commercially available blood collection devices and the testing device used to assay the samples.

Thus, there is a need for improved patient-centric sample collection devices that also provide automated and better integration with testing equipment (e.g., automated testing equipment).

### SUMMARY

Accordingly, the present disclosure relates to collection devices with features adaptable to collect samples such as biological fluids samples (e.g., blood or bodily fluids in humans or animals). In some embodiments, the collection device as described herein is automation compatible. In some embodiments, the collection device as described herein enables easy transport to a testing facility, and/or broader adoption of medical and home testing (e.g., collection of samples from home) with capillary blood samples as compared with conventional sample collection devices. In some embodiments, the collection device as described herein is used for point of care testing with a variety of devices. In some embodiments, a collection device that provides easy sample collection is critical to enable the expansion of tests that can be performed in this settings using capillary blood to replace venous blood.

As described herein, the samples for research, applied markets, diagnostics, and/or other tests (e.g., antigen testing, hormone level testing, genetic testing, pathogen detection by molecular, antibody-based methods and other methods, vaccination status, antibody level testing, blood smears, complete blood count (CBC), hemoglobin, hematocrit, electrolyte panel, neonatal blood gasses, neonatal bilirubin, neonatal screening, glucose, lipids (cholesterol, HDL cholesterol, LDL cholesterol), A1C levels, blood glucose levels, triglycerides, infectious diseases, sexually transmitted diseases, other disease state testing, and/or all other blood tests) can be collected from any biological anatomy suitable for collection. For example, the samples can be blood samples, saliva samples, mucus samples, and/or other biological fluids samples. The samples in the present disclosure can be collected using a capillary (e.g., a complete tube capillary, a three-sided capillary, a parallel structure capillary or other suitable capillaries), fiber paper, or foam matrix, and/or other structures or devices capable of collecting and retaining, drying and/or transferring fluids as needed. In general, the collected samples can be used to perform any suitable assay or test, e.g., blood smears, complete blood count (CBC), hemoglobin, hematocrit, electrolyte panel, neonatal blood gasses, neonatal bilirubin, neonatal screening, glucose, lipids (cholesterol, HDL cholesterol, LDL cholesterol), triglycerides, infectious diseases, sexually transmitted diseases, and/or all other blood tests or other sample collections.

Various embodiments of the collection devices as described herein include a capillary that can be combined with or replaced by other types of collection structures (e.g., lancets, brushes, swabs, or any suitable collection devices). In various embodiments, a collection structure as described herein include substantially similar size, shape, components, methods of use, manufacturing steps, and/or other features with another collection device as described herein. For example, in the present disclosure, a capillary as described herein may include substantially the same length with a brush as descried herein. In another example, a capillary including a three-sided structure may share similar methods in use with a capillary including two parallel planes. In various embodiments, a collection structure as described herein include a different size, shape, component, methods of use, manufacturing steps, and/or other features with another collection device as described herein.

Advantageously, the collection device can include a capillary or other types of collection structures (e.g., lancets, brushes, swabs), configured to interface with automation equipment used in a testing facility (e.g., decapping machine, transport robotics, shaking apparatus, etc.) for automated processing (e.g., decapping, sample testing, or other processing steps). Furthermore, the capillary is operatively attached to the automation compatible cap, where the cap is either removable or locked with the capillary. Following collection of a sample (e.g., performed by a medical personnel or by a patient), the capillary or other collection structure containing the sample can be inserted into a vial, the cap can be coupled to the vial, and the vial can be transported to an automation system or devices to perform automated processing (e.g. decapping, sample testing, or other processing steps) or can be used directly on a point of care device at the location of sample collection. Thus, such collection device may provide a convenient, efficient, and cost-effective apparatus, system and method for transporting and automated testing of samples.

Additionally, easier serum or plasma separations, at the sight of the patient or when the whole blood sample returns to the laboratory, along with these more efficient integrations into the diagnostic workflows, can enable wider adoption of capillary blood collection across broader clinical laboratory and business applications.

In some embodiments, the present disclosure includes air drying of the sample, blood drying devices, systems or methods (e.g., for drying the blood in the cap or vial). In some embodiments, the present disclosure include devices, systems or methods (e.g., surface treatment or other physical methods) to separate blood components at the point of collection and in other cases, separation of whole blood into components after the sample returns to the laboratory.

Disclosed herein is a collection device, comprising: a capillary for collecting samples from a biological location of a body; and a cap configured to interface with an automation device.

In some embodiments, the capillary comprises a cross-section of one or a plurality of U-shapes, V-shapes, L-shapes, double L shapes, curved shapes, polygonal shapes.

In some embodiments, the capillary comprises one or more sides, planes, overhangs, or combinations thereof.

In some embodiments, the capillary comprises a three-sided structure.

In some embodiments, the capillary comprises a parallel structure.

In some embodiments, the capillary is adapted to collect biological fluids samples.

In some embodiments, the capillary is a complete capillary.

In some embodiments, the capillary is an incomplete capillary.

In some embodiments, the cap is adapted to be secured to a vial prior to interfacing with the automation device.

In some embodiments, the cap comprises a threaded portion. In some embodiments, the threaded portion is configured to interface with a threaded portion of the vial for sealing the vial.

In some embodiments, the automation device comprises at least one of a decapper machine and a handheld device.

In some embodiments, the collection device further comprises one or more channels configured to improve sample travelling through the one or more channels.

In some embodiments, the collection device further comprises one or more indicators at or near a distal end of the capillary and attached to the surface of the capillary, wherein the one or more indicators are configured to provide a visual signal indicating that the sample collected in the capillary reaches an indicator line.

In some embodiments, the collection device further comprises a capillary tip operatively coupled to a proximal end of the capillary, wherein the capillary tip is configured to transfer the samples into one or more channels.

In some embodiments, the collection device further comprises an assembly feature configured to accommodate assembly of a sample collection medium into the cap for collection of the samples.

In some embodiments, the sample collection medium comprises an FTA® paper.

In some embodiments, the assembly feature couples the capillary to the cap operatively and firmly to provide a final collection device using any one of press-fit, glue, ultrasonic welding, rivet, or combinations thereof.

In some embodiments, the collection device is configured to separate the different components of blood.

In some embodiments, the collection device comprises a suction device operatively coupled to either or both ends of the cap to aid in collecting the samples by suctioning.

In some embodiments, the suction device is operatable by an automated machine.

Additionally disclosed herein is a sample collection device for collecting samples from a biological location of a body, comprising: a support portion; and a brush head extending substantially perpendicular from a surface of the support portion, wherein the brush head comprises a plurality of sample collection bristles configured to include a plurality of sampling sheets, such that the bristles are conveniently adhered to a sample collection area of the biological location of the body.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIGS. 1A-1E are images of an exemplary automation compatible collection device including a complete capillary, according to various embodiments;

FIGS. 2A-2C are images of various exemplary automation compatible collection devices including incomplete capillaries with various structures, according to various embodiments;

FIGS. 3A-3C are images of an exemplary automation compatible collection device including an incomplete capillary structure, according to various embodiments;

FIGS. 4A-4D are images of another exemplary automation compatible collection device including an incomplete capillary structure, according to various embodiments;

FIGS. 5A-5D are images of yet another exemplary automation compatible collection device including an incomplete capillary structure, according to various embodiments;

FIGS. 6A-6C are images of yet another exemplary automation compatible collection device including an incomplete capillary structure, according to various embodiments;

FIGS. 7-14 are schematic views of various exemplary automation compatible collection devices, according to various embodiments;

FIGS. 15-24 are schematic, detail views of various additional features of exemplary automation compatible collection devices, according to various embodiments;

FIGS. 25A and 25B are schematic, detail views of an exemplary brush of a sample collection device, according to various embodiments;

FIG. 26 shows schematic perspective views of an exemplary automation compatible collection device manufactured by injection molding, according to various embodiments;

FIG. 27 shows images of exemplary materials for manufacturing a collection device, according to various embodiments; and

FIGS. 28A and 28B are images of an exemplary 96-well rack that can be used with vials containing samples using a collection device, according to various embodiments.

### DETAILED DESCRIPTION

Various embodiments of the present invention are directed to automation compatible collection devices that are better adapted to collect samples than prior collection devices. Various components and features of the collection device, according to various embodiments, are described in greater detail below. This application will often describe the technology as directed to an automation compatible collection device.

In some embodiments, the systems and methods as described herein include any type of collection device, capillary or other types of devices, with or without a cap. For example, the system and methods as described herein can be applied to swabs, lancets, cuvettes, and other known devices for collecting samples. In some embodiments, the collection device in the present disclosure includes a stem that includes a capillary (e.g., complete and/or incomplete capillary), other collection structures, or combinations thereof. For example, the entire stem of the collection device can include a capillary. In another example, a portion of the stem (e.g., a portion at and/or closer to the proximal end of the stem for collecting samples) of the collection device can include a capillary, while another portion of the stem (e.g., a portion at and/or closer to the distal end of the stem operatively attached to the cap) of the collection device can include other structures such as a rod.

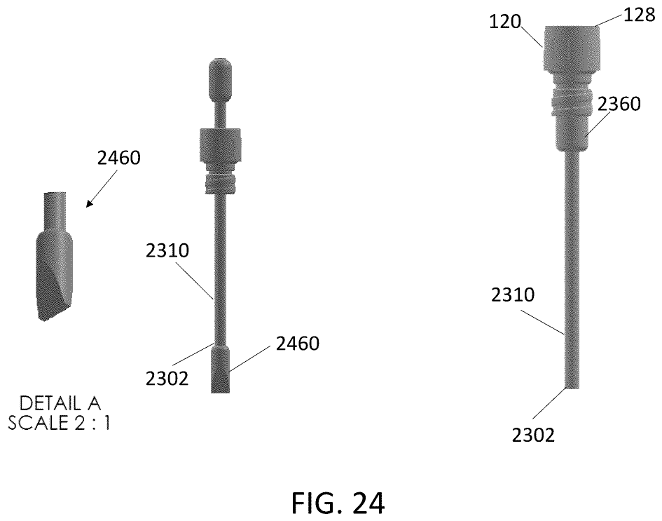

In some embodiments, the collection device in the present disclosure includes mechanical devices, such as a suction device (FIG. 23 or 24), a duck-bill valve (FIG. 24), and/or other suitable devices to obtain fluids. For example, the mechanical device can generate a pressure gradient to pull or draw fluids using e.g., suction, pumping, and/or vacuum mechanisms.

### Collection Device

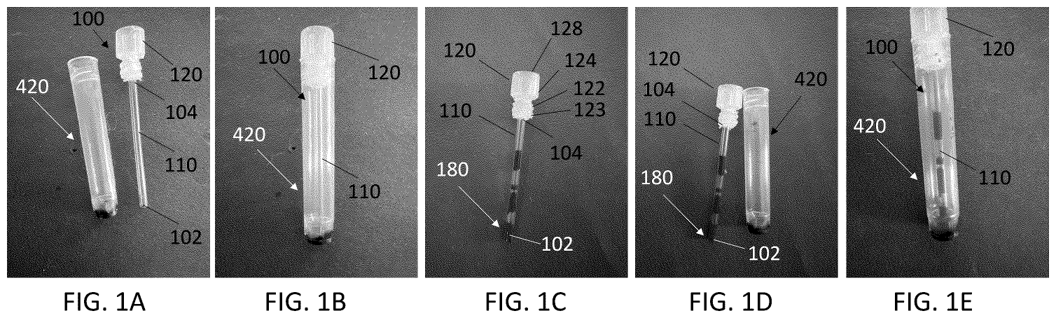

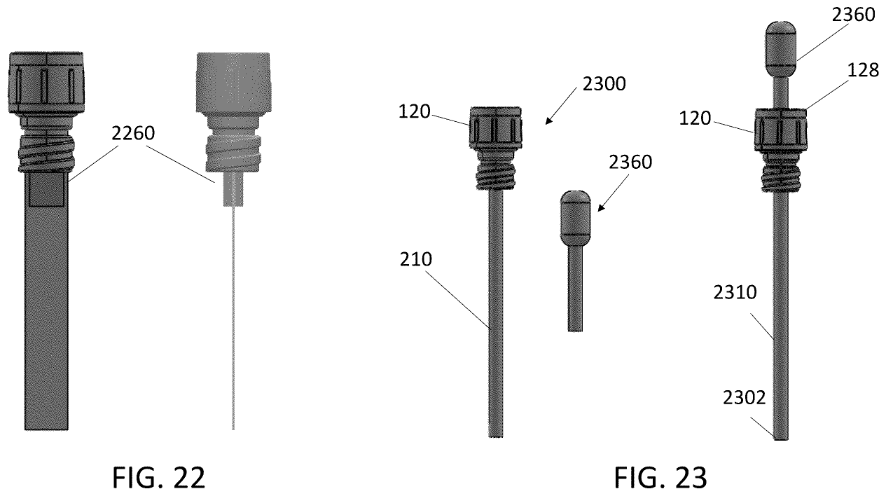

In general, the collection device as described herein (e.g., collection devices as shown in FIGS. 1-25) can be used to collect any suitable biological fluids (e.g., blood from a vein) at any biological location (e.g., finger, arms, legs, or other suitable regions) of a subject (e.g., a body of a human or an animal, etc.).

In some embodiments, the collection device is configured to collect a sample for research, tests, and/or any other uses from sample collections.

In some embodiments, the collection device is configured to perform tests (e.g., for analysis and/or detection) on the subject.

In some embodiments, the tests include antigen testing, hormone level testing, genetic testing, vaccination status testing, exposure to pathogens (or viruses, bacteria) testing, antibody level testing (e.g., for analysis of immune status), interferon and/or metabolites (e.g., natural and synthetic metabolites) testing (e.g. for detecting infections such as tuberculosis (TB), or cancer), blood smears, complete blood count (CBC), hemoglobin, hematocrit, electrolyte panel, neonatal blood gasses, neonatal bilirubin, neonatal screening, glucose, lipids (cholesterol, HDL cholesterol, LDL cholesterol), A1C levels, blood glucose levels (e.g., for monitoring or detection of diabetes), triglycerides, infectious diseases, sexually transmitted diseases, other disease state testing, and/or all other blood tests. In particular embodiments, the tests include a blood test. In some embodiments, the tests include a dried blood spot test on e.g., absorbent paper, foam matrix or other materials. In some embodiments, the tests can include serum, plasma or whole blood as the starting matrices depending on the need from a test menu.

In some embodiments, the collection device is configured to collect a sample for determination of physiological and/or biochemical states, such as disease, mineral content, pharmaceutical drug effectiveness, and organ function.

In some embodiments, the collection device is configured to collect a sample from a subject infected with or suspected to be infected with a sexually transmitted disease (STD). In some embodiments, the STDs include at least one of chlamydia, genital herpes, genital warts or human papillomavirus, gonorrhea, hepatitis A, hepatitis B, hepatitis C, syphilis, trichomoniasis, human immunodeficiency virus (HIV), cytomegalovirus, molluscum contagiosum, Mycoplasma genitalium, bacterial vaginosis, or others.

In some embodiments, the collection device is configured to collect a sample from a subject infected with or suspected to be infected with an infection detectible in blood of a subject. In some embodiments, the subject is infected with or suspected to be infected with an infection at hormone levels, A1c and blood glucose levels (e.g., for monitoring or detection of diabetes), genetic levels, antibody levels (e.g., for analysis of immune status), or antigen levels. In some embodiments, the subject is infected with or suspected to be infected with tuberculosis (TB).

In some embodiments, the collection device is automation compatible.

In some embodiments, the collection device, vial, and/or their components (e.g., capillary, cap, and any portions of these) are disposable, reusable, biodegradable, compostable, and/or recyclable.

In some embodiments, the collection device, vial, and/or their components (e.g., capillary, cap, and any portions of these) are configured to include a smooth surface without abrasive or sharp features, in order to ensure safe use of the collection device and to avoid damage to the subject or injury to the sample collection operator.

In some embodiments, changes to the surface are achieved by surface treatments and/or treatment with chemicals (e.g., cellulose acetate, heparin, EDTA, anticoagulents, or other suitable chemicals) and in some cases antibodies and other components that aid in specific component separations

In some embodiments, the surface can contain materials that act as the actual assay to provide a clinical results.

In some embodiments, at least part of the surface treatment can allow separation of blood or other fluid samples into different components.

In some embodiments, the surface treatment includes cross hatching, oxidation, mechanical abrasion, mechanical machining, mechanical texturing, chemical reaction, chemical etching, plasma or other high energy reactions, surface coating with one or more materials homogeneously or heterogeneously, and/or other suitable treatment methods.

In some embodiments, the surface treatment changes the surface properties of the collection device, such as the hydrophilicity, chemical reactivity, and/or biological reactivity (e.g., coagulation) of the collection device.

In some embodiments, the collection device, vial, and/or their components (e.g., capillary, cap, and any portions of these) includes features or elements (e.g., desiccation, anticoagulants, or other suitable elements) adjust the speed at which the collected sample fluid dries or coagulates in the collection device.

In some embodiments, the collection device, vial, and/or their components (e.g., capillary, cap, and any portions of these) are compatible with standard wells for 1536-well plate formats, 384-well plate formats, 96-well plate formats, 48-well plate formats, 36-well plate formats, 24-well plate formats, 12-well plates formats, any other standard well-plate formats. In particular embodiments, the devices, systems, and methods as described herein include a universal size of well formats. In particular embodiments, the systems and methods as described herein include 24-well plate formats, 48-well plate formats, and/or any desired formats. In particular embodiments, the vial described herein in the present disclosure include a diameter ranging from about 5 mm to about 20 mm, from about 10 mm to about 15 mm, or is about 13 mm. In particular embodiments, the vial described herein in the present disclosure include a height ranging from about 50 mm to about 100 mm, from about 60 mm to about 80 mm, or is about 76 mm.

In some embodiments, the devices, systems, and methods as described herein do not need a well plate format. For example, testing (or analysis) of a sample that is collected by the collection device as described herein can be performed directly off the collection device. In another example, testing (or analysis) of a sample that is collected by the collection device as described herein can be performed using a vial (or tube) that has a different size (e.g., a smaller diameter) than a vial (or tube) in a standard well-plate format. In another example, testing (or analysis) of a sample that is collected by the collection device as described herein may involve reading results directly from a sample without an elution step, such as testing a calorimetric change in the sample by applying a reagent.

In general, the sample collection device in the present disclosure can take any known form, such as a complete capillary, an incomplete capillary, a brush (e.g., a cervical brush), other forms or structures, or their combinations thereof. In some embodiments, the sample collection device is coupled to or separate from other elements (e.g., a cap) for the collection of samples, as described below in further detail.

In some embodiments, a portion (e.g., a portion at and/or closer to the proximal end of the stem for collecting samples) of the collection device includes a first structure (e.g., a complete capillary and/or an incomplete capillary), while another portion (e.g., a portion at and/or closer to the distal end of the stem operatively attached to the cap) of the collection device includes another structure (e.g., a brush, a rod, a stem, or other suitable structures) that is the same or different from the first structure.

In some embodiments, the collection device in the present disclosure includes a stem that includes a proximal end to collect samples from a sample area, and a distal end that is operatively attached to the cap on a distal end. In some embodiments, the stem of the collection device includes a capillary (complete or incomplete), other collection structures, or combinations thereof. In some embodiments, the entire stem of the collection device can be a same structure (e.g., a capillary). In some embodiments, a portion of the stem (e.g., a portion at and/or closer to the proximal end of the stem for collecting samples) of the collection device includes a capillary, while another portion of the stem (e.g., a portion at and/or closer to the distal end of the stem operatively attached to the cap) of the collection device includes other structures such as a rod.

In some embodiments, the sample collection device does not need to include a capillary. For example, the sample collection device can include any one of an absorbent paper, fibers, foams, or other materials or structures, which can be attached to an automation compatible cap.

In some embodiments, the capillary or other structure can include any configuration that is sufficient to collect a sample from a subject or for the subject to collect their own sample in a decentralized testing model.

In some embodiments, the capillary includes one or more complete capillaries or structures, one or more incomplete capillaries, or their combinations thereby. For example, in some embodiments, the collection device includes an incomplete capillary at the proximal end, and a complete capillary at the distal end. In another example, in some embodiments, the collection device includes an incomplete capillary at the distal end, and a complete capillary at the proximal end. In another example, the capillary includes one or more capillaries disposed within a larger capillary.

In some embodiments, at least part of the capillary or other structure is of an absorbent head type. For example, the capillary or other structure can be flocked, foam, spun fibers, etc. In some embodiments, the capillary or other structure is coated by at least one absorbent material (e.g., flocked materials, plant-derived materials, animal-derived materials, synthetic materials, foams, a combination of these, or other suitable materials). In some embodiments, the capillary or other structure is non-absorbent (e.g., not flocked, not coated by fibrous materials).

In some embodiments, the collection device includes a porous collection zone (e.g., channel or interior of a capillary).

In some embodiments, the collection device includes a non-porous collection zone (e.g., channel or interior of a capillary).

In some embodiments, mechanical methods (e.g., methods using suction or pressure differences) are used to collect, retain, or transfer fluids.

Complete Capillary

FIGS. 1A-1E are images of an exemplary collection device 100, according to various embodiments. FIGS. 1A and 1B are perspective views of the collection device 100 and an exemplary vial 420 for receiving the collection device 100 e.g., before collecting biological fluids. FIGS. 1C-1E are perspective views of the collection device 100 and the vial 420 including collected biological fluids (e.g., blood 180).

In some embodiments, at least part of the capillary 110 is a complete capillary such as a pipette or a capillary tube.

In some embodiments, the collection device (e.g., collection device 100 or 200) in the present disclosure includes a capillary and/or a cap. In some embodiments, the collection device in the present disclosure includes 2, 3, 4, 5, or more capillaries, caps, absorbent papers, fibers, foams, and/or other materials or structures. In some embodiments, the capillary 110 is separate from or not coupled to a cap 120. In some embodiments, the cap 120 is operatively, removably, and/or firmly coupled (e.g., attached) to the capillary 110. In some embodiments, the cap is irremovable and locked with the capillary 110. In some embodiments, the cap 120 can be adapted to be operatively coupled to any collection device (e.g., blood collection device, swabs, and/or other sample collection devices from any part of a biological location of a subject).

In some embodiments, the capillary 110 can include an axial shaft (e.g., a hollow cylindrical rod). In some embodiments, the capillary 110 includes a proximal end 102 and a distal end 104. In some embodiments, the proximal end 102 and/or the distal end 104 include any suitable shape, e.g., rounded, pointed, grooved, and/or other suitable shapes.

In some embodiments, the capillary 110 is configured to be open at one or both ends (e.g., at the proximal end 102 and/or the distal end 104) of the capillary 110. Such configuration can enable collecting samples from a sample collection location (e.g., vessels or capillary beds at fingertips or other parts of the body) and/or connecting other devices (e.g., a cap, lancet, needle, intravenous line or PICC line, tube, additional capillary, a finger-stick capillary bed, microfluidics device, point of care device, an automated device, or other suitable devices or elements). Accordingly, in use (e.g., in a hospital), the capillary 110 can be connected to other devices for processing samples collected by the capillary 110.

In some embodiments, the capillary 110 includes a length (e.g., distance between the first proximal end 102 and the distal end 104) in a range of about 5 mm to about 200 mm, about 10 mm to about 175 mm, about 15 mm to about 150 mm, about 20 mm to about 125 mm, about 25 mm to about 100 mm, about 30 mm to about 75 mm, or about 35 mm to about 50 mm. For example, the capillary 110 can include a length of about any of: 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 110 mm, 120 mm, 130 mm, 140 mm, 150 mm, 160 mm, 170 mm, 180 mm, 190 mm, or 200 mm.

In some embodiments, the capillary 110 includes an outer diameter compatible with standard wells for 1536-well plate formats, 384-well plate formats, 96-well plate formats, 48-well plate formats, 36-well plate formats, 24-well plate formats, 12-well plates formats, or any other standard well-plate formats.

In some embodiments, the capillary 110 includes an outer diameter in a range from about 0.1 mm to about 6 mm, about 0.5 mm to about 5.5 mm, about 1.0 mm to about 5 mm, about 1.5 mm to about 4.5 mm, about 2 mm to about 4 mm, about 2.5 mm to about 3.5 mm, about 1 mm to about 50 mm, about 5 mm to about 45 mm, about 10 mm to about 40 mm, about 15 mm to about 35 mm, about 20 mm to about 30 mm. In some embodiments, the capillary 110 includes an outer diameter of about any of: 0.1 mm, 0.5 mm, 1 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, 15 mm, 20 mm, 25 mm, 30 mm, 35 mm, 40 mm, 45 mm, or 50 mm. In some embodiments, the capillary 110 includes an outer diameter of less than about any of: 0.1 mm, 0.5 mm, 1 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, 15 mm, 20 mm, 25 mm, 30 mm, 35 mm, 40 mm, 45 mm, or 50 mm.

In some embodiments, the capillary 110 includes the same outer diameter for the entirety of the capillary 110. In various embodiments, the capillary 110 includes at least two different outer diameters for the entirety of the capillary 110. For example, the diameter of a first portion (e.g., the proximal end 102) of the capillary 110 may be different from the diameter of a second portion (e.g., the distal end 104) of the capillary 110.

In some embodiments, the capillary 110 is a complete capillary and includes a hollow interior that is surrounded (e.g., fully surrounded as shown in FIG. 1) by the inner wall of the capillary 110. In some embodiments (e.g., FIG. 1), the inner wall of the capillary 110 defines an inner diameter (e.g., diameter of the hollow interior) of the capillary 110 in a range from about 0.1 mm to about 6 mm, about 0.5 mm to about 5.5 mm, about 1.0 mm to about 5 mm, about 1.5 mm to about 4.5 mm, about 2 mm to about 4 mm, about 2.5 mm to about 3.5 mm, about 1 mm to about 50 mm, about 5 mm to about 45 mm, about 10 mm to about 40 mm, about 15 mm to about 35 mm, about 20 mm to about 30 mm. In some embodiments, the hollow interior of the capillary 110 includes an outer diameter of about any of: 0.1 mm, 0.5 mm, 1 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, 15 mm, 20 mm, 25 mm, 30 mm, 35 mm, 40 mm, 45 mm, or 50 mm. In some embodiments, the capillary 110 includes an outer diameter of less than about any of: 0.1 mm, 0.5 mm, 1 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, 15 mm, 20 mm, 25 mm, 30 mm, 35 mm, 40 mm, 45 mm, or 50 mm.

In some embodiments, the capillary 110 includes the same inner diameter for the entirety of the capillary 110. In various embodiments, the capillary 110 includes at least two different inner diameters for the entirety of the capillary 110. For example, the diameter of a first portion of the hollow interior (e.g. at the proximal end 102) of the capillary 110 may be different from the diameter of a second portion of the hollow interior (e.g., at the distal end 104) of the capillary 110.

In some embodiments, the capillary 110, or at least a portion of the capillary 110 tapers (e.g., has sequentially reduced outer diameters and/or inner diameters) towards the proximal end 102, the distal end 104, from the middle of the capillary 110 or any point of the capillary 110.

In some embodiments, the capillary 110 tapers from a maximum diameter at the proximal end 102 of the capillary 110 to a minimum diameter at the distal end 104 of the capillary 110.

In some embodiments, the capillary 110 tapers from a maximum diameter at the distal end 104 of the capillary 110 to a minimum diameter at the proximal end 102 of the capillary 110.

In some embodiments, the maximum diameter of the capillary 110 occurs at any point of the capillary 110 and the diameters taper to a minimum diameter at the proximal end 102 and/or the distal end 104 of the capillary 110.

In some embodiments, the minimum diameter of the capillary 110 occurs at any point of the capillary 110 and the diameters taper to a maximum diameter at the proximal end 102 and/or the distal end 104 of the capillary 110.

In some embodiments, the diameter of the capillary 110 alternates between a minimum diameter and a maximum diameter from the proximal end 102 to the distal end 104 of the capillary 110.

In some embodiments, the capillary 110 (outer surface and/or hollow interior) can include a cross-section of at least one shape of a polygonal shape, e.g., a triangle, a square, a quadrilateral, a trapezoid, a pentagon, a hexagon, a polygon with at least 7, 8, 9, 10, 11, 12, 13, 14, 15, 16, 17, 18, 19, 20 or more sides, a circle, a circle with one or more flat sides, an ellipse, or an ellipse with one or more flat sides. In some embodiments, at least one side of the cross-section includes a convex and/or concave curve. In some embodiments, the cross-section includes a rotationally symmetric shape or an asymmetric shape. In some embodiments, the cross-section includes the same shape, or at least two different shapes for the entirety of the component.

In some embodiments, the performance of the complete capillary (e.g., capillary 110) can be improved by adapting the length of the capillary, dimensions, and/or structures of the capillary.

Incomplete Capillary or Open Capillary

In some embodiments, the capillary is an incomplete capillary or open capillary (e.g., capillary 210).

Advantageously, an incomplete or open capillary as described herein can result in improved performance (e.g., in comparison with a closed or complete capillary), as described below.

In some embodiment, the open capillary can facilitate wicking of samples (e.g., blood) and/or drying of samples (e.g., blood) for transport, increase patient comfort, and/or safety.

In some embodiment, an open capillary can improve ease of use by increasing visibility of collected samples when collecting the samples. For example, the blood being collected in opaque capillaries can be visualized to a patient or physician. Accordingly, a desired volume of collected blood can be achieved.

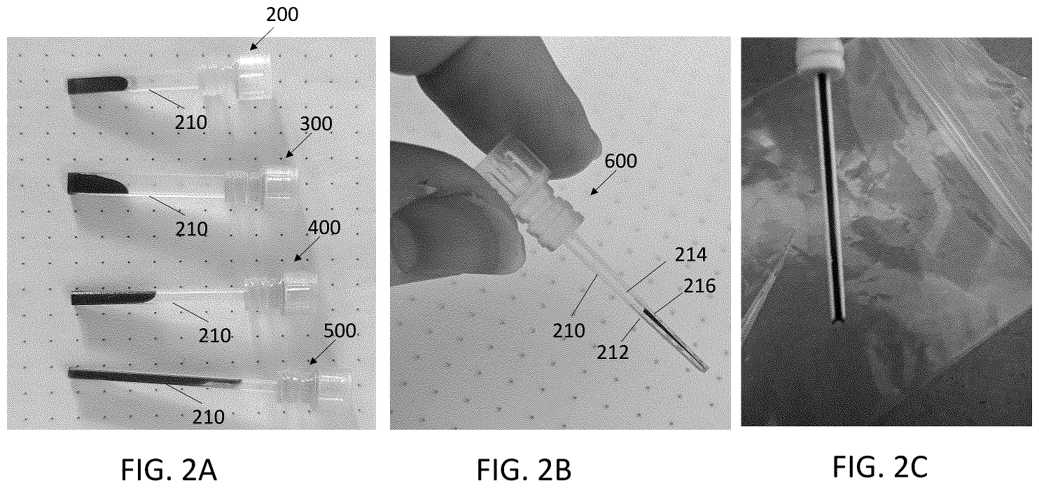

In some embodiment, an open capillary can result in easier and improved elution of samples. For example, such improve elution of blood can enable separation of blood into various components (e.g., red blood cell (RBC), white blood cell (WBC), and/or plasma), filter potential contaminants, and thus allowing optical tests. In another example, the blood can be collected through the entire length of capillary (e.g., FIG. 2C), which can result in more rapid and complete sample elution and thus better assay performance.

In some embodiment, an open capillary can prevent bubbles during collection.

In some embodiment, an open capillary can provide or facilitate access to surface treatment of e.g., a molding surface or final part to modify surface properties;

In some embodiment, an open capillary can provide improved manufacturability. For example, it can enable injection mold of a single component as the final product, without the need to extrude a closed or complete capillary to assemble to a cap.

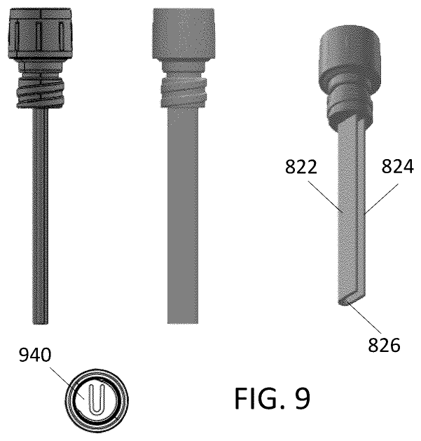

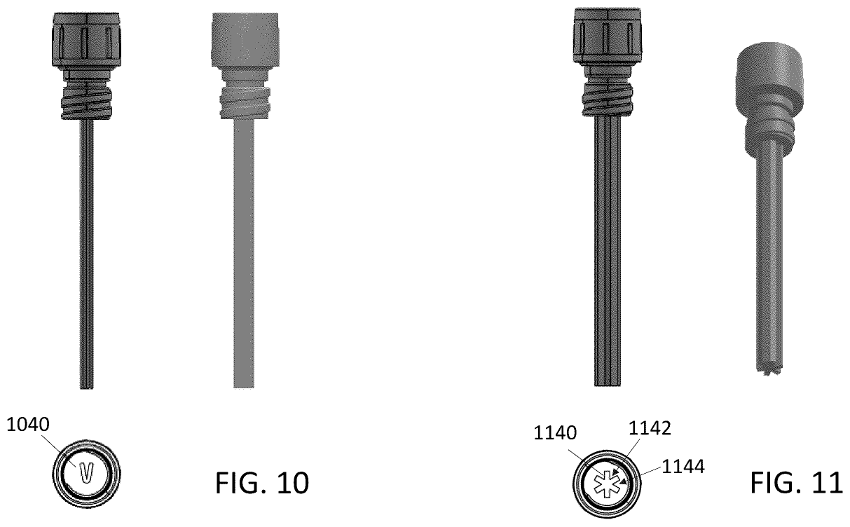

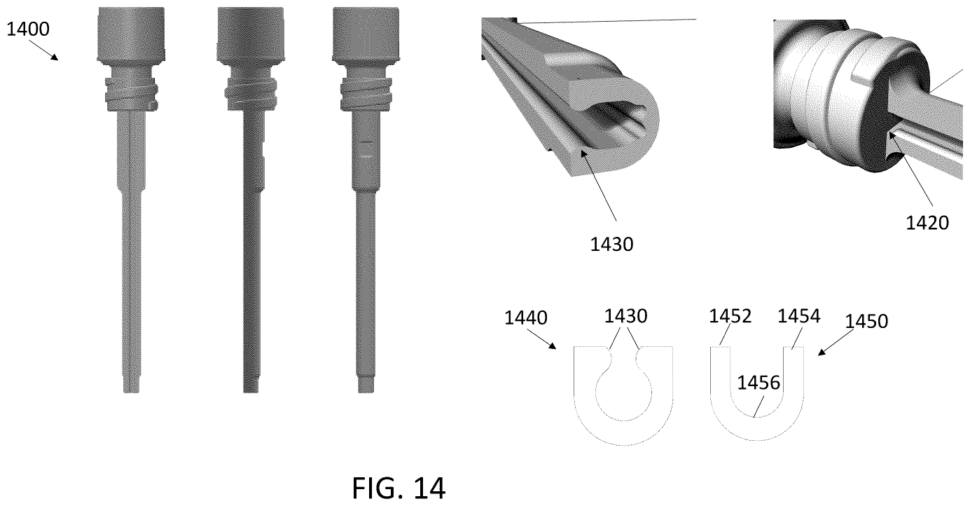

Thus, the sample collected by the incomplete capillary may be exposed to the environment (e.g., air). In some embodiments, the incomplete capillary includes a cross-section of one or a plurality of U-shapes (e.g., as shown in FIG. 2A, 4, 5, 6, or 9), V-shapes (e.g., as shown in FIG. 10), L-shapes (not shown), double L shapes (e.g., as shown in FIG. 2A, 2B, or 3), curved (e.g., semi-circle, truncated circle, semi-ellipse, truncated ellipse) shapes, polygonal shapes, or other suitable shapes with 1, 2, 3, or more sides or planes (e.g., flat or grooved sides or planes) (e.g., as shown in FIGS. 9, 11-14), 1, 2, or more overhangs (e.g., overhangs as shown in FIG. 14), and/or any combinations thereof.

In some embodiments, the cross-section of the incomplete capillary can be at least one of an incomplete circle, U-shape, V-shape, double L shape, curved shape, polygonal shape, or other suitable shapes or forms with about 5% to about 95%, about 5% to about 50%, about 10% to about 90%, about 15% to about 85%, about 20% to about 80%, about 25% to about 75%, about 30% to about 70%, about 35% to about 65%, about 40% to about 60%, about 45% to about 55%, of the circle or other shapes in closed forms missing).

In some embodiments, at least one side of the incomplete capillary is configured to include one or more convex curves, concave curves, gaps, holes, openings, or their combinations along the side of the capillary. Such configuration can increase exposure of the capillary to the environment and/or improve access for elution.

In some embodiments, the cross-section of the incomplete capillary includes a rotationally symmetric shape or an asymmetric shape.

In some embodiments, the cross-section of the incomplete capillary includes the same shape, or at least two different shapes for the entirety of the incomplete capillary.

As shown in FIGS. 2-6, the collection devices 200, 300, 400, 500, or 600 includes a capillary 210 and/or a cap 120. In some embodiments, the capillary 210 is separate from or not coupled to a cap 120. In some embodiments, a cap 120 is operatively, removably, and/or firmly coupled (e.g., attached) to the capillary 210. In some embodiments, the cap 120 can be adapted to be operatively coupled to any collection device (e.g., blood collection device, swabs, and/or other sample collection devices from any part of a biological location of a subject).

In some embodiments, the capillary 210 includes a proximal end 202 and a distal end 204. In some embodiments, the proximal end 202 and/or the distal end 204 include any suitable shape, e.g., rounded, pointed, grooved, and/or other suitable shapes.

In some embodiments, the capillary 210 is configured to be open at one or both ends (e.g., at the proximal end 202 and/or the distal end 204) of the capillary 210. Such configuration can enable collecting samples from a sample collection location (e.g., vessels or capillary beds at finger tips or other parts of the body) and/or connecting other devices (e.g., a cap, lancet, needle, intravenous line or PICC line, tube, additional capillary, a finger-stick capillary bed, an automated device, or other suitable devices or elements).

In some embodiments, the capillary 210 includes a length (e.g., distance between the first proximal end 202 and the distal end 204) in a range of about 1 mm to about 1000 mm, about 2 mm to about 800 mm, about 3 mm to about 600 mm, about 4 mm to about 400 mm, about 5 mm to about 200 mm, about 10 mm to about 175 mm, about 15 mm to about 150 mm, about 20 mm to about 125 mm, about 25 mm to about 100 mm, about 30 mm to about 75 mm, or about 35 mm to about 50 mm. For example, the capillary 210 can include a length of at least about 1 mm, 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 110 mm, 120 mm, 130 mm, 140 mm, 150 mm, 160 mm, 170 mm, 180 mm, 190 mm, 200 mm, 300 mm, 400 mm, 500 mm, 600 mm, 700 mm, 800 mm, 900 mm, or 1000 mm.

In some embodiments, a side of the incomplete capillary 210 includes a thickness, width, or height in a range of about 0.1 mm to 0.5 mm, about 0.5 mm to about 50 mm, about 1 mm to about 45 mm, about 1.5 mm to about 40 mm, about 2 mm to about 35 mm, about 2.5 mm to about 35 mm, about 3 mm to about 30 mm, about 3.5 mm to about 25 mm, about 4 mm to about 20 mm, about 4.5 mm to about 15 mm, or about 5 mm to about 10 mm. For example, at least one of the sides 312, 314, and 316 can include a length of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm.

In some embodiments, the thickness, width, or height of at least one side of the capillary 210 is the same through the entire length of the capillary (e.g. from a proximal end 202 to a distal end 204 of the capillary 210). In some embodiments, the capillary 210 includes at least two different thicknesses, widths, or heights through the entire length of the capillary (e.g. from a proximal end 202 to a distal end 204 of the capillary 210).

In some embodiments, two sides or planes of the incomplete capillary 210 includes a spacing (e.g., maximum spacing, minimum spacing, medium spacing, average spacing, or other spacing parameters) in a range of about 0.1 mm to about 50 mm, about 1 mm to about 45 mm, about 1.5 mm to about 40 mm, about 2 mm to about 35 mm, about 2.5 mm to about 35 mm, about 3 mm to about 30 mm, about 3.5 mm to about 25 mm, about 4 mm to about 20 mm, about 4.5 mm to about 15 mm, or about 5 mm to about 10 mm. For example, the distance between the first portion 422 and the second portion 424 can include a length of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm. In some embodiments, the spacing is in a range of about 0.1 mm to about 0.5 mm, about 0.1 mm to about 0.4 mm, about 0.2 mm to about 0.4 mm, or about 0.2 mm to about 0.3 mm. In some embodiments, the spacing is about 0.1, 0.2, 0.3, 0.4, or 0.5 mm.

In some embodiments, the spacing is the same through the entire length of the capillary (e.g. from a proximal end 202 to a distal end 204 of the capillary 210). In some embodiments, the capillary 210 includes at least two different spacings through the entire length of the capillary.

In some embodiments, the incomplete capillary 210 includes at least one channel between two sides or planes for storing or transferring the collected samples.

In some embodiments, the channel includes a width in a range of about 0.5 mm to about 50 mm, about 1 mm to about 45 mm, about 1.5 mm to about 40 mm, about 2 mm to about 35 mm, about 2.5 mm to about 35 mm, about 3 mm to about 30 mm, about 3.5 mm to about 25 mm, about 4 mm to about 20 mm, about 4.5 mm to about 15 mm, or about 5 mm to about 10 mm. For example, the distance between the first portion 422 and the second portion 424 can include a length of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm. In some embodiments, the channel includes a width in a range of about 0.1 mm to about 0.5 mm, about 0.1 mm to about 0.4 mm, about 0.2 mm to about 0.4 mm, or about 0.2 mm to about 0.3 mm. In some embodiments, the channel includes a width of about 0.1, 0.2, 0.3, 0.4, or 0.5 mm.

In some embodiments, the channel includes a depth in a range of about 0.5 mm to about 50 mm, about 1 mm to about 45 mm, about 1.5 mm to about 40 mm, about 2 mm to about 35 mm, about 2.5 mm to about 35 mm, about 3 mm to about 30 mm, about 3.5 mm to about 25 mm, about 4 mm to about 20 mm, about 4.5 mm to about 15 mm, or about 5 mm to about 10 mm. For example, the distance between the first portion 422 and the second portion 424 can include a length of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm. In some embodiments, the channel includes a depth in a range of about 0.1 mm to about 0.5 mm, about 0.1 mm to about 0.4 mm, about 0.2 mm to about 0.4 mm, or about 0.2 mm to about 0.3 mm. In some embodiments, the channel includes a depth of about 0.1, 0.2, 0.3, 0.4, or 0.5 mm.

In some embodiments, the width of the channel is the same through the entire length of the capillary (e.g. from a proximal end 202 to a distal end 204 of the capillary 210). In some embodiments, the capillary 210 includes at least two different widths of the channel through the entire length of the capillary.

In some embodiments, the width of the channel is greater than the depth of the channel. In some embodiments, the width of the channel is greater than the depth of the channel throughout the channel. In some embodiments, the width and the depth of the channel are configured such that the channel includes a Urchin structure.

In some embodiments, the width of the channel is smaller than the depth of the channel. In some embodiments, the width of the channel is smaller than the depth of the channel throughout the channel.

In some embodiments, the capillary can hold a volume of samples in a range of about 0.1 ml to 20 ml, about 0.5 ml to 15 ml, about 1 ml to 10 ml, about 2 ml to 9 ml, about 3 ml to 8 ml, about 4 ml to 7 ml, or about 4 ml to 6 ml. In some embodiments, the channel can hold a volume of samples of at least about 0.1 ml, 0.2 ml, 0.3 ml, 0.4 ml, 0.5 ml, 0.6 ml, 0.7 ml, 0.8 ml, 0.9 ml, 1 ml, 1.5 ml, 2 ml, 2.5 ml, 3 ml, 3.5 ml, 4 ml, 4.5 ml, 5 ml, 5.5 ml, 6 ml, 6.5 ml, 7 ml, 7.5 ml, 8 ml, 8.5 ml, 9 ml, 9.5 ml, 10 ml, 11 ml, 12 ml, 13 ml, 14 ml, 15 ml, 16 ml, 17 ml, 18 ml, 19 ml, or 20 ml.

In some embodiments, the curved portion of the incomplete capillary 210 includes a diameter (e.g., maximum, minimum, medium, or average diameter) in a range of about 0.5 mm to about 50 mm, about 1 mm to about 45 mm, about 1.5 mm to about 40 mm, about 2 mm to about 35 mm, about 2.5 mm to about 35 mm, about 3 mm to about 30 mm, about 3.5 mm to about 25 mm, about 4 mm to about 20 mm, about 4.5 mm to about 15 mm, or about 5 mm to about 10 mm. For example, the curved shape 532 includes a diameter of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm.

In some embodiments, the diameter of the curved portion is the same through the entire length of the capillary (e.g. from a proximal end 202 to a distal end 204 of the capillary 210). In some embodiments, the capillary 210 includes at least two different diameters of the curved portion through the entire length of the capillary.

In various embodiments, the length, width, or height of at least one side or plane of the capillary tapers from a maximum at the proximal end 202 of the capillary 210 to a minimum at the distal end 204 of the capillary 210. In some embodiments, as shown in FIG. 2B, the distance between two sides 212 and 214 (or the width of the side 216) tapers from a maximum at the proximal end 202 of the capillary 210 to a minimum at the distal end 204 of the capillary 210.

In various embodiments, the length, width, or height of at least one side or plane of the capillary tapers from a maximum at the distal end 204 of the capillary 210 to a minimum at the proximal end 202 of the capillary 210.

In various embodiments, the maximum of such characteristics of the capillary 210 occurs at any point of the capillary 210 and taper to a minimum at the proximal end 202 and/or the distal end 204 of the capillary 210.

In various embodiments, the minimum of such characteristics occurs at any point of the capillary 210 and taper to a maximum at the proximal end 202 and/or the distal end 204 of the capillary 210.

In various embodiments, such characteristics of the capillary 210 alternates between a minimum and a maximum from the proximal end 202 to the distal end 204 of the capillary 210.

FIGS. 2A and 2B are images of various exemplary automation compatible collection devices 200, 300, 400, 500, and 600, according to various embodiments.

As shown in FIGS. 2A and 2B, in some embodiments, at least part of the collection device (e.g., 200, 300, 400, 500, or 600) includes an incomplete capillary 210, 310, 410, 510, or 610. In some embodiments, the incomplete capillary 210, 310, 410, 510, and/or 610 includes a cross section that can take any known form or structure with 1, 2, 3, or more sides. For example, the incomplete capillary 210 includes a three-sided structure (e.g., a cross section including a double L form with three sides). In another example, the incomplete capillary 310 includes a parallel-plane structure (e.g., a cross section including a U-shape form including two parallel planes). In another example, the incomplete capillaries 410 and 510 include a curved (e.g., semi-circle or semi-ellipse) shape. polygonal (e.g., triangle) shape. In some embodiments, the cross-section of the incomplete capillary 210, 310, 410, 510, or 610 includes the same shape through the entire length of the capillary (e.g. from a proximal end to a distal end of the capillary). In some embodiments, the cross-section of the incomplete capillary 210, 310, 410, 510, or 610 includes at least two different shapes through the entire length of the capillary (e.g. from a proximal end to a distal end of the capillary).

In some embodiments, the capillary 210 or any of its components is compatible with standard wells for 1536-well plate formats, 384-well plate formats, 96-well plate formats, 48-well plate formats, 36-well plate formats, 24-well plate formats, 12-well plates formats, any other standard well-plate formats, any desired size (e.g., a universal size) of well formats, or any combination thereof.

FIG. 2C is an image of an exemplary automation compatible collection device including an incomplete capillary structure. As shown in FIG. 2C, the incomplete capillary demonstrates a successful performance in collecting blood through substantially the entire length of the incomplete capillary structure.

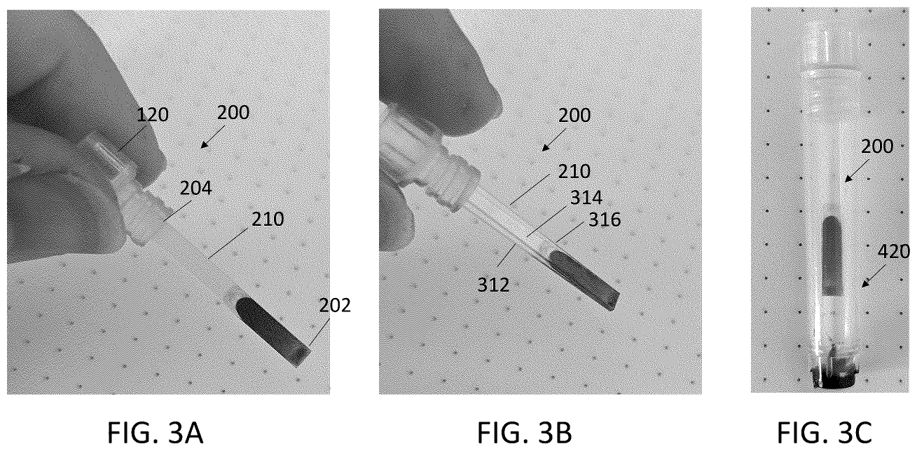

FIGS. 3A-3C are images of an exemplary automation compatible collection device 200. In some embodiments, the compatible collection device 200 includes an incomplete capillary 210 that is three-sided. For example, as shown in FIG. 3B, the capillary 210 includes a first side 312, a second side 314, and a third side 316.

In some embodiments, at least one of the sides 312, 314, and 316 of the incomplete capillary 210 includes a thickness, width, height, or spacing with respect to another side in a range of about 0.5 mm to about 50 mm, about 1 mm to about 45 mm, about 1.5 mm to about 40 mm, about 2 mm to about 35 mm, about 2.5 mm to about 35 mm, about 3 mm to about 30 mm, about 3.5 mm to about 25 mm, about 4 mm to about 20 mm, about 4.5 mm to about 15 mm, or about 5 mm to about 10 mm. For example, at least one of the sides 312, 314, and 316 can include a length of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm.

In some embodiments, the thickness, width, height, or spacing is the same through the entire length of the capillary (e.g. from a proximal end 202 to a distal end 204 of the capillary 210). In some embodiments, capillary includes at least two different thicknesses, widths, heights, or spacings through the entire length of the capillary.

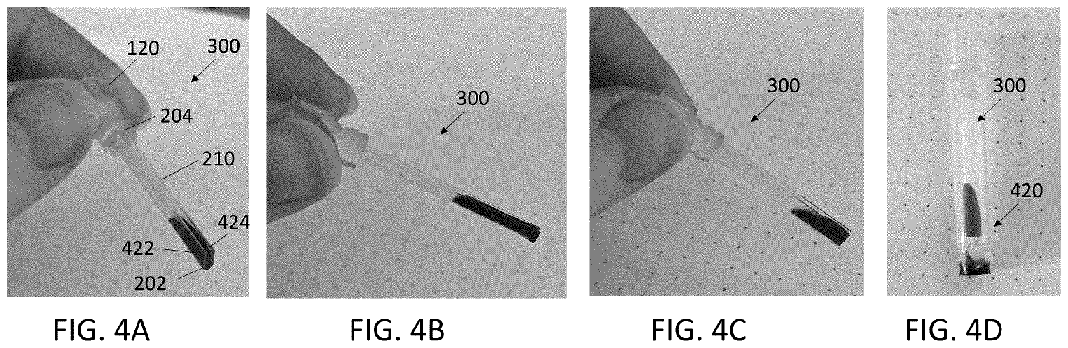

FIGS. 4A-4D are images of another exemplary automation compatible collection device 300. In some embodiments, the compatible collection device 300 includes an incomplete capillary 210 in a curved structure.

As shown in FIG. 4A, the capillary 210 includes a first portion 422 and a second portion 424. In some embodiments, the first portion 422 and a second portion 424 are parallel such that the incomplete capillary 210 is in a parallel form or structure. Accordingly, the first portion 422 and the second portion 424 are configured to have the same spacing throughout the height (e.g., the distance between the top and the bottom of the cross section) of the incomplete capillary 210. In some embodiments, the first portion 422 and a second portion 424 can be configured to form any nonparallel structure of the capillary 210. Accordingly, the first portion 422 and the second portion 424 are configured to have at least two different spacings throughout the height (e.g., the distance between the top and the bottom of the cross section) of the incomplete capillary 210.

In some embodiments, the spacing (e.g., maximum spacing, minimum spacing, medium spacing, or average spacing) between the first portion 422 and the second portion 424 is in a range of about 0.5 mm to about 50 mm, about 1 mm to about 45 mm, about 1.5 mm to about 40 mm, about 2 mm to about 35 mm, about 2.5 mm to about 35 mm, about 3 mm to about 30 mm, about 3.5 mm to about 25 mm, about 4 mm to about 20 mm, about 4.5 mm to about 15 mm, or about 5 mm to about 10 mm. For example, the distance between the first portion 422 and the second portion 424 can include a length of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm.

In some embodiments, the distance between the first portion 422 and the second portion 424 is the same through the entire length of the capillary (e.g. from a proximal end 202 to a distal end 204 of the capillary 210). In some embodiments, the distance between the first portion 422 and the second portion 424 tapers through the entire length of the capillary (e.g. from a proximal end 202 to a distal end 204 of the capillary 210. In some embodiments, the distance between the first portion 422 and the second portion 424 alternates through the entire length of the capillary (e.g. from a proximal end 202 to a distal end 204 of the capillary 210). In some embodiments, the maximum and/or minimum distance between the first portion 422 and the second portion 424 occurs at any point through the entire length of the capillary (e.g. from a proximal end 202 to a distal end 204 of the capillary 210).

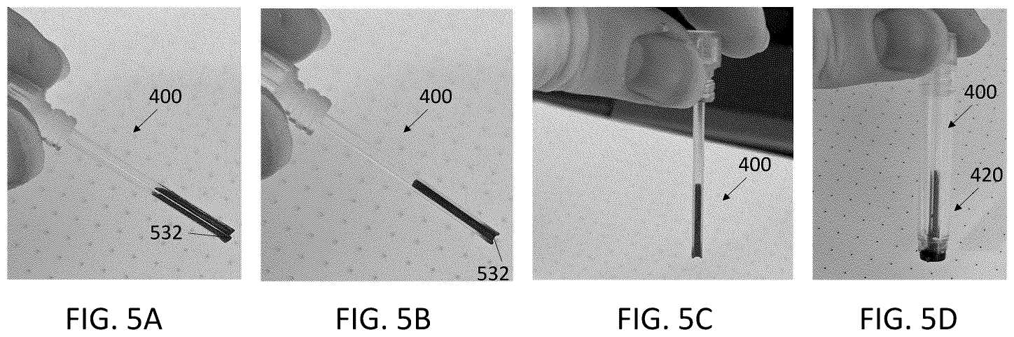

FIGS. 5A-5D are images of another exemplary automation compatible collection device 400. In some embodiments, the compatible collection device 400 includes an incomplete capillary 210 in a curved structure.

As shown in FIG. 5A-5C, the cross section of the incomplete capillary 210 includes a curved shape 532 (e.g., semi-circle).

In some embodiments, the curved shape 532 includes a diameter (e.g., maximum, minimum, medium, or average diameter) in a range of about 0.5 mm to about 50 mm, about 1 mm to about 45 mm, about 1.5 mm to about 40 mm, about 2 mm to about 35 mm, about 2.5 mm to about 35 mm, about 3 mm to about 30 mm, about 3.5 mm to about 25 mm, about 4 mm to about 20 mm, about 4.5 mm to about 15 mm, or about 5 mm to about 10 mm. For example, the curved shape 532 includes a diameter of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm.

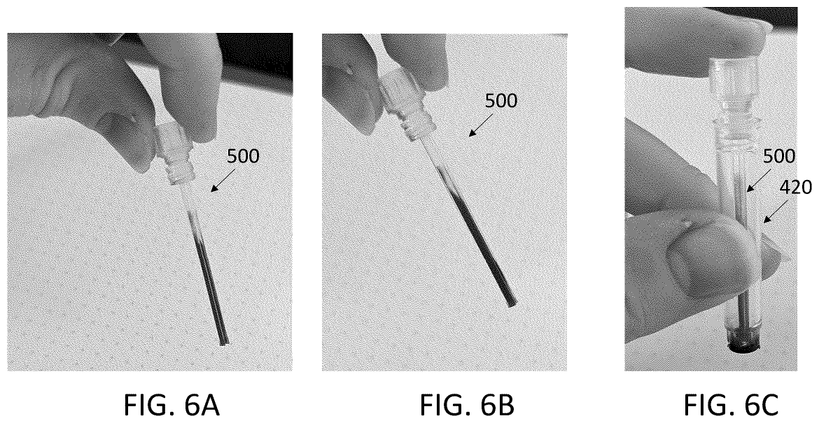

FIGS. 6A-6C are images of another exemplary automation compatible collection devices. As compared with FIGS. 6A-6C, the length of the capillary 210 is longer, and the curved portion 532 includes a smaller diameter.

In some embodiments, the performance of the complete capillary (e.g., capillary 110) can be improved by adapting the length of the capillary, dimensions and/or structures of the capillary.

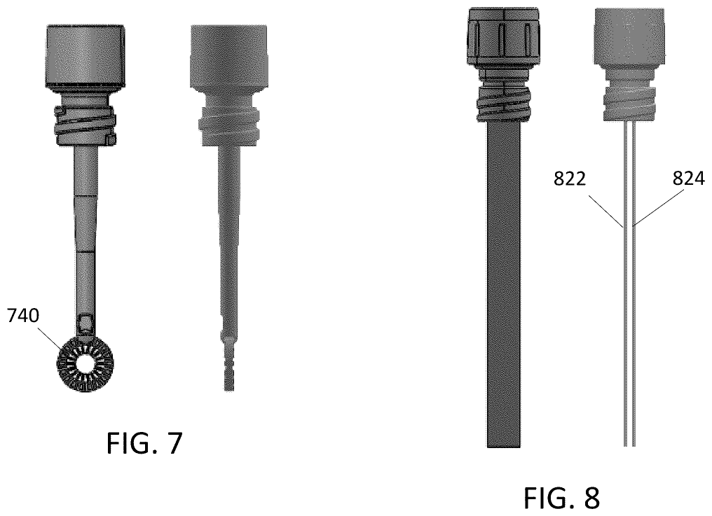

FIGS. 7-14 are schematic views of various exemplary automation compatible collection devices, according to various embodiments. In particular embodiments, the automation compatible collection device, as shown in FIGS. 7-14, includes an automation compatible cap (e.g., cap 120) and a capillary (e.g., capillary 110 or 210) including various structures, as described herein.

Referring to FIG. 7, in some embodiments, the incomplete capillary includes one or more cavities (e.g., cavities 740). In some embodiments, the cavities are configured to form a loop at or near the proximal end of the capillary (e.g., the end away from the cap). In some embodiments, the cavities are open to at least one side of the incomplete capillary. In some embodiments, the cavities include features to capture blood inside at least one cavity. In some embodiments, such features can allow fast drying of blood for transport, and/or other improved performance as described herein.

Referring to FIGS. 8 and 9, in some embodiments, the incomplete capillary includes parallel plates (e.g., plates 822 and 824 in FIGS. 8 and 9) to wick (or absorb) sample into the channel (e.g., channel 826) between the plates. Such parallel plates results in a three-sided U-shape cross-section (e.g., from a bottom view 940) of the capillary). In some embodiments, the capillary in FIGS. 8 and 9 includes the same structure as the capillary as discussed in FIGS. 4A-4D. Accordingly, a volume of samples collected can be determined by the width of each plate and the distance between the two plates.

Referring to FIG. 10, in some embodiments, the incomplete capillary includes a cross-section of V-shape (e.g., V-shape cross-section 1040). Such cross-section results in a V-shaped channel that can wick (or absorb) sample into the sample collection device. Accordingly, a volume of samples collected can be determined by the width or depth of the channel.

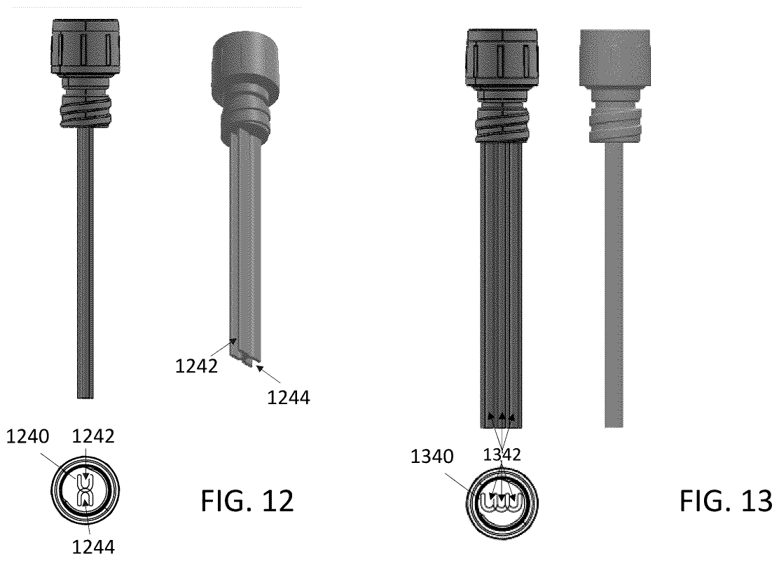

In some embodiments, as shown in FIGS. 11-13, the incomplete capillary includes a plurality of plates, a plurality of sides, and/or a cross-section including various shapes, resulting in multiple cavities on the outside surface of the capillary. Accordingly, a volume of samples collected can be determined by the width or depth of the multiple cavities. For example, as shown in FIG. 11, the cross-section of the incomplete capillary includes a plurality of V-shapes (e.g., multi-V shaped cross-section 1140 in FIG. 11. Such configuration can wick (or absorb) sample into the cavities (e.g., cavities 1142 and 1144) along the outside surface of the device e.g., via capillary action. In another example, as shown in FIG. 12, the cross-section (e.g., cross-section 1240) of the incomplete capillary includes two U-shape structures. Such configuration can wick (or absorb) sample into the cavities (e.g., cavities 1242 and 1244) along the outside surface of the device e.g., via capillary action. In another example, as shown in FIG. 13, the cross-section (e.g., cross-section 1340) of the incomplete capillary includes three U-shape structures. Such configuration can wick (or absorb) sample into the cavities (e.g., cavities 1342) along the outside surface of the device e.g., via capillary action.

In some embodiments, the sample collection device includes one or more overhangs located at interior surface of the sample collection device. In general, the overhangs can improve performance of the collection device such as facilitating transport of the collected sample from one end to the other end of the sample collection device.

FIG. 14 shows views of a sample collection device 1400 including a capillary 1410 including overhangs, and vies of cross sections (e.g., cross sections 1440 and 1450) of the sample collection device 1400. In some embodiments, the capillary 1410 is an incomplete capillary that has a cross section 1450 including a U-shape form and three sides (or surfaces) 1452, 1454, and 1456. In some embodiments, two sides 1452 and 1454 are located at or in proximity to the two end portion of the cross section 1450 of the sample collection device 1400, and/or the side 1456 is located at the interior surface of the cross section 1450. In some embodiments, the sample collection device 1400 includes an overhang 1430 at or in proximity to the interior surface of each end portion of the sample collection device 1400. In some embodiments, the overhang 1430 includes any shape or form (e.g., a smooth outer surface). In some embodiments, overhangs 1430 may be generated for a round, 3-sided, and/or open capillary. In some embodiments, an overhang is configured to stop (e.g., during de-molding step) at a short distance (e.g., from about 0.1 mm to about 50 mm) from the intersection (e.g., intersection 1420 as shown in FIG. 14) of the cap and the capillary to allow the final product to flex and release from the mold.

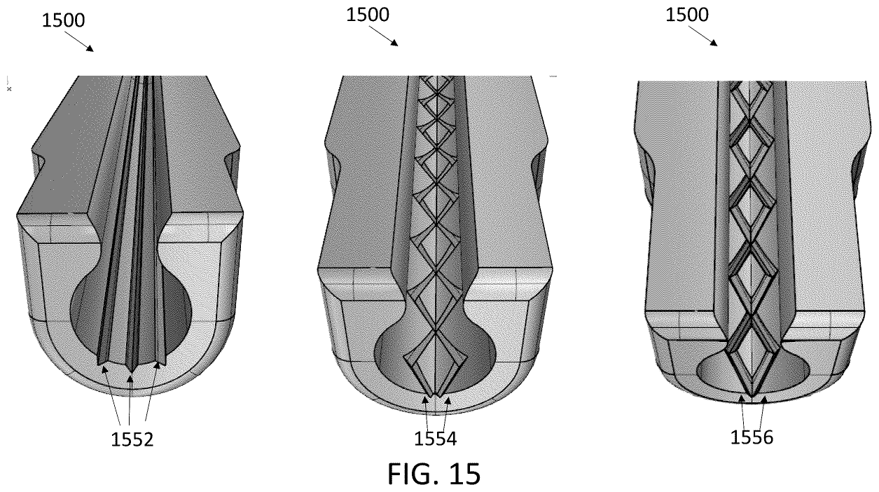

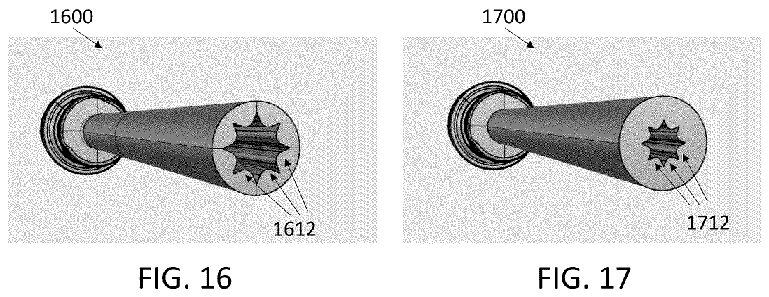

FIGS. 15-17 shows collection devices including one or more sub-channels. In some embodiments, the sub-channels include textures to improve sample travelling through the channel, improve performance of sample collections, and/or facilitate transport of the collected samples. In general, the sub-channels can include any structures or configurations. As shown in FIG. 15, in some embodiments, the interior surface of the incomplete capillary includes a grooved or textured surface including sub-channels (or secondary channels) (e.g. sub-channels 1552, 1554, or 1556) located at the interior surface of an incomplete capillary 1500. As shown in FIGS. 16 and 17, in some embodiments, a complete capillary 1600 or 1700 includes one or more sub-channels 1612 or 1712 located at the internal surface of the capillary 1600 or 1700. Accordingly, the capillary 1600 or 1700 includes one or more textured channels with uneven surfaces in the interior of the capillary 1600 or 1700. In general, a sub-channel can include any desired depth, width, and/or length. For example, a sub-channel can start from one end of the capillary to the other end of capillary, and thus may include a substantially same length as the capillary. In another example, a sub-channel includes a distance from one or both ends of the capillary, and thus may include a shorter length than the length of the capillary. In another example, a sub-channel (e.g., sub-channel 1612 as shown in FIG. 16) may include a deeper groove or texture than another sub-channel (e.g., sub-channel 1712 as shown in FIG. 17).

Cap

In some embodiments, the collection device further includes a cap (e.g., cap 120 in FIGS. 1-6). In some embodiments, the cap is configured to be attached to one end (e.g., the distal end) of the capillary (e.g., capillary in FIGS. 1-14). In some embodiments, the cap is operatively, removably, and/or firmly coupled (e.g., attached) to the capillary. In some embodiments, the cap is irremovable and locked with the capillary. In some embodiments, the cap or a portion of the cap can be formed as a single molded part (e.g., a unitary part or item) and/or as separate parts. In some embodiments, the cap can be used as a handle by the person using the collection device (e.g., before the collection device is broken and shortened). In some embodiments, the cap is adapted to fit any standard or custom vial and/or compatible to any automation process.

In some embodiments, the cap includes interior or exterior features (e.g., ring or bubble blower, urchin structures) configured to collect and/or hold fluids.

In some embodiments, the cap is adapted to any collection device (e.g., capillary as described herein, swab, lancet, or other suitable devices).

In some embodiments, the cap comprises a structure and/or configuration adapted to interface with an automation device (e.g., a tube capper or decapper machine). In general, the cap can have any structure that corresponds with any known or future developed automation device (e.g., manufactured by Rhinostics, Azenta, Hamilton, Tecan, Abbott™, ThermoFisher™ Roche, Hologic, etc.). For example, in some embodiments, the cap comprises a hollow internal portion, e.g., that interfaces with an automated device. In some embodiments, an outer surface of the cap (e.g., top surface, circumferential surface, side surface) interfaces with an automation device. In some embodiments, the proximal end of the cap (i.e., farther away from the proximal end) defines an opening leading to hollow internal portion of the cap. In some embodiments, the cap comprises a hollow cylinder. In some embodiments, the cap is defined by an outer cross-section (i.e., the external shape of the cap) and an inner cross-section (i.e., the internal shape of the hollow portion). In some embodiments, the outer and/or inner cross-section of the cap is a circle, a semicircle, a truncated circle, or a circle with one or more flat sides. In some embodiments, the outer and/or inner cross-section of the cap is a circle. In some embodiments, the outer and/or inner cross-section of the cap comprises a polygonal cross section, e.g., a cross-section in the shape of a triangle, a square, a quadrilateral, a trapezoid, a pentagon, a hexagon, a star, or a polygon with at least 7, 8, 9, 10, 11, 12, 13, 14, 15, 16, 17, 18, 19, 20 or more sides. In some embodiments, at least one side of the outer and/or inner cross-section of the cap comprises a convex and/or concave curve. In some embodiments, the outer and/or inner cross section of the cap is a rotationally symmetric shape. In some embodiments, the outer and/or inner cross section of the cap is an asymmetric shape. In some embodiments, the outer and/or inner cap cross-section is the same for the entirety of the cap. In some embodiments, the outer and/or inner cap cross-section is different for at least one portion of the cap; the cap can comprise any combination of different (e.g., at least 2, at least 3, at least 4, at least 5) cap cross-sections. In some embodiments, the outer and inner cap cross-sections of the cap are the same. In some embodiments, the outer and inner cap cross-sections of the cap are different.

In some embodiments, the cap comprises at least one (e.g., 1, 2, 3, 4, 5, or more) internal groove(s). In some embodiments, the cap comprises at least one (e.g., 1, 2, 3, 4, 5, or more) internal ridge(s). In some embodiments, the cap comprises at least one (e.g., 1, 2, 3, 4, 5, or more) external groove(s). In some embodiments, the cap comprises at least one (e.g., 1, 2, 3, 4, 5, or more) external ridge(s). In some embodiments, the internal or external groove(s) or internal ridge(s) are parallel with the axial shaft of the collection device.

In some embodiments, the cap can interface with an automated device. In some embodiments, the automated device can move, control, manipulate, etc. the collection device after interfacing with the cap. In some embodiments, a portion of an automated device can extend into the hollow internal portion of the cap. In some embodiments, hollow portion and the internal groove(s) or internal ridge(s) permit the cap to interface with an automated device. In some embodiments, the automated device is a tube capper and decapper machine. In some embodiments, the cap can be adjusted to fit any standard or custom tube that is compatible with the SBS 24-well format, the SBS 48-well format, the SBS 96-well format, any desired size (e.g., a universal size) of well formats, or any combination thereof. In some embodiments, the cap can be adjusted for any automation format. In particular embodiments, the devices, systems, and methods as described herein include a universal size of well formats. In particular embodiments, the systems and methods as described herein include 24-well plate formats, 48-well plate formats, and/or any desired formats. In particular embodiments, the vial described herein in the present disclosure include a diameter ranging from about 5 mm to about 20 mm, from about 10 mm to about 15 mm, or is about 13 mm. In particular embodiments, the vial described herein in the present disclosure include a height ranging from about 50 mm to about 100 mm, from about 60 mm to about 80 mm, or is about 76 mm.

In some embodiments, the cap does not need to interface with a well plate format. In some embodiments, the devices, systems, and methods as described herein do not need a well plate format. For example, testing (or analysis) of a sample that is collected by the collection device as described herein can be performed directly off the collection device. In another example, testing (or analysis) of a sample that is collected by the collection device as described herein can be performed using a vial (or tube) that has a different size (e.g., a smaller diameter) than a vial (or tube) in a standard well-plate format. In another example, testing (or analysis) of a sample that is collected by the collection device as described herein may involve reading results directly from a sample without an elution step, such as testing a calorimetric change in the sample by applying a reagent.

In some embodiments, the capillary as described herein does not interfere with interaction between the cap and the automation device. For example, upon in use, the hollow interior of cap would be unimpeded by the capillary.

In some embodiments, the cap is removably, operatively, and/or firmly coupled (or connected, attached) to the capillary or other collection devices and can be freely detached from the capillary.

In some embodiments, the cap 120 is translatable along the capillary or other collection devices. For example, the cap 120 can move from one position to another position between the proximal end and distal end of the capillary. In some embodiments, the cap 120 is configured to stay attached to (e.g., locked at) the capillary (e.g., before or after the sample is collected). In some embodiments, after the collection device is inserted into a vial, the cap 120 stays attached to the capillary in the vial (e.g., a vial 420 in FIGS. 1 and 3-6). In some embodiments, during an automation process (e.g., decapping), the cap 120 is configured to be detached from the capillary 100, in response to an application of an external force from e.g., an automated device as described herein.

In some embodiments, the cap may include a threaded portion 122 (e.g., a cylindrical portion including one or more raised helical threads). The threaded portion 122 is configured to interface with a threaded portion of a vial (or container tube) for sealing the cap 120 with the vial.

In some embodiments, the threaded portion 122 is hollow, and the proximal end of the threaded portion 122 defines an opening leading to an hollow internal portion in the threaded portion 122 of the cap 120. Thus, the threaded portion 122 of the cap 120 can receive and operatively coupled to the capillary.

In some embodiments, the threaded portion 122 includes at least 1, 2, 3, 4, or 5 threads 123. In some embodiments, the threads 123 are external threads located on the outside of the threaded portion 122. In other embodiments, the threads 123 are internal threads located inside the threaded portion 122. In some embodiments, the threads 123 are continuous, discontinuous, or a combinations thereof.

In some embodiments, the threaded portion 122 includes a geometry, pitch, direction, number, and/or dimensions of the threads that matches the threaded portion of the vial such that the cap 120 can be fully, firmly and/or securely screw in place to the vial.

In some embodiments, the threaded portion 122 includes an inner diameter (e.g., a diameter of the opening) and/or an outer diameter, which are substantially equal to or greater than the average diameter of the capillary. In some embodiments, the inner diameter and/or the outer diameter of the threaded portion 122 is from about 0.5 mm to about 10 mm.

In some embodiments, the cap 120 includes an annular portion 124 (e.g., a cylindrical portion) attached to the threaded portion 122. When the cap is attached to the capillary, the threaded portion 122 is closer to the incomplete capillary than the annular portion 124.

In some embodiments, the annular portion 124 is hollow, and the proximal end of the annular portion 124 defines an opening 128 leading to an hollow internal portion of the annular portion 124 of the cap 120. Thus, the annular portion 124 of the cap 120 can receive and operatively coupled to the capillary. In some embodiments, the annular portion 124 includes an inner diameter (e.g., a diameter of the opening 128) and/or an outer diameter. In some embodiments, the inner diameter and/or outer diameter of the annular portion 124 is greater than the diameter of the capillary, the diameter of the opening of the cap 120, and/or the outer parameter of the threaded portion 122. In some embodiments, the inner diameter and/or the outer diameter of the annular portion 124 is from about 0.05 mm to about 10 mm. In some embodiments, the outer diameter of the annular portion 124 defines the maximum diameter of the cap 120. In some embodiments, the annular portion 124 includes a solid interior (e.g., not hollow).

In some embodiments, the cap may be locked and stay attached to the capillary without having the mobility (e.g., along any direction). Upon being acted upon by an external force (e.g., from a decapper machine, manual device, or other suitable automation devices) the cap can be removed from the vial. In some embodiments, the cap is configured to remain attached to the capillary upon being acted upon by an automation device (e.g., such that upon removal of the cap from the vial, so is the capillary). In such instances, a testing process can occur using either the cap attached to the capillary (e.g., by transporting the cap/capillary into an assay solution) and/or a testing process can occur using the material left behind in the vial (e.g., with the cap and capillary removed from the vial, the assay solution can be delivered into the vial). In other embodiments, the automation device can detach the cap from the capillary, such that the capillary remains behind in the vial while the assay solution is delivered. In either embodiment, following the application of an assay solution (or other testing protocol), the cap can either be resecured to the vial for storage/incubation of the tests or the cap can be disposed of and a different cap can be used for storage/incubation.

In some embodiments, the cap is configured to interface with an automation device (e.g., a decapper machine). For example, the cap includes at least one (e.g., 1, 2, 3, 4, 5, or more) internal ribs to enable an operation by an automation device. In some embodiments, the internal ribs are parallel with the axial shaft of the capillary. In some embodiments, the internal ribs includes a height less than or equal to the height of the annular portion of the cap. In some embodiments, the internal ribs includes a diameter less than the inner diameter of the annular portion of the cap.

In some embodiments, the cap is configured to be attached to the capillary firmly or securely in place, by applying an antirotation feature (not shown). In some embodiments, the antirotation feature is located at the inner wall of the annular portion, or any suitable position within the cap.

In some embodiments, the cap can be adjusted to fit any standard or custom vial that is compatible with the SBS 24-well format, the SBS 48-well format, the SBS 96-well format, or any combination thereof. In some embodiments, the cap can be adjusted for any automation format.

In some embodiments the caps is supplied separate from the capillary. In some embodiments, the cap is supplied with the capillary.

Additional Features or Elements

In some embodiments, the collection device includes one or more additional features or elements, such as one or more textured channels, sub-channels, indicators, capillary tips, and cap capillary assembly features, as described herein. In some embodiments, the term “channel” refers to the “cavities” as shown in e.g., FIGS. 8-11.

In some embodiments, the capillary (e.g., complete capillary or incomplete capillary) of the collection device includes one or more channels (e.g., channels or cavities as shown in FIGS. 8-11), or sub-channels (e.g., textures or grooves as shown in FIGS. 15-17). The channels and/or sub-channels include textures to improve sample travelling through the channel, improve performance of sample collections, and/or facilitate transport of the collected samples. For example, as shown in FIG. 15, the channel texturing include one or more sub-channels (or secondary channels) (e.g. sub-channels 1552, 1554, or 1556). In some embodiments, the sub-channel can include any structures or configurations. In some embodiments, as shown in FIGS. 16 and 17, a complete capillary includes one or more sub-channels located at the internal surface of the capillary such that the capillary includes a textured channel with uneven surfaces in the interior.

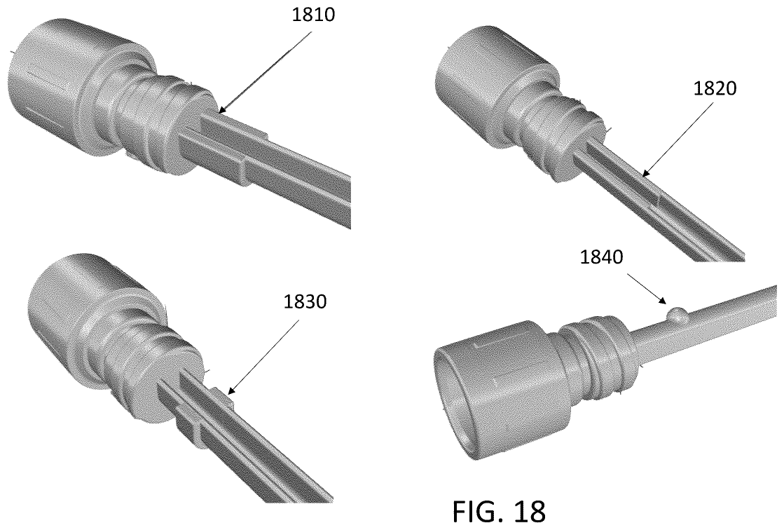

Referring to FIG. 18, in some embodiments, the collection device includes one or more indicators (e.g., volume indicators) at or near the distal end (the end closer to the cap) of the capillary. The indicators can provide a visual signal indicating that the sample collected in the capillary reaches the indicator line. Thus, the indicators can alert a user when a target volume of collected fluid is achieved. In some embodiments, the indicator can be an element 1810 or 1830 attached to the outside surface of the capillary, where the element 1810 extends to the intersection of the cap and the collection device and the element 1830 ends at a short distance from the intersection of the cap and the collection device. In some embodiments, the indicator can be an element 1820 attached to the inside surface of the capillary. In some embodiments, the indicator extends to the cap. In some embodiments, the indicator can be a protuberance 1840 located at the outside surface of the capillary. In some embodiments, the indicator can include any configurations.

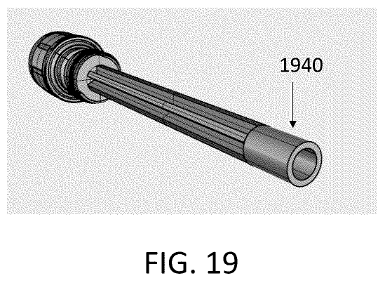

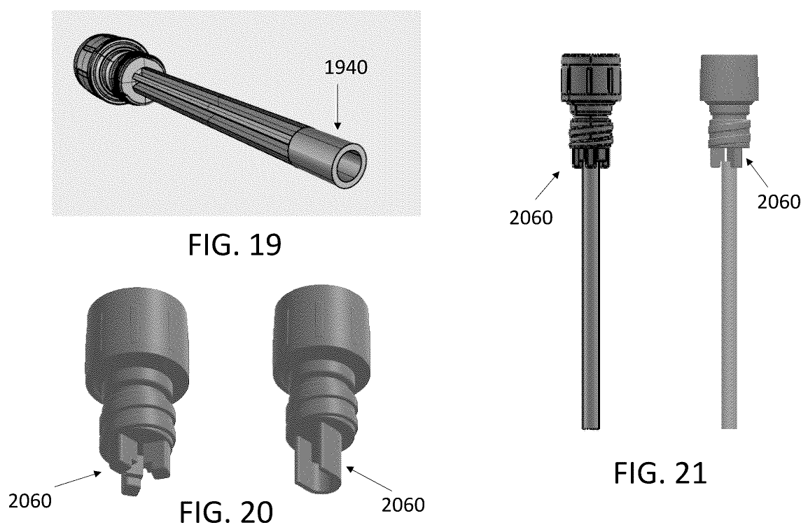

Referring to FIG. 19, in some embodiments, the collection device further includes a capillary tip (e.g., capillary tip 1940) operatively coupled to the proximal end of the capillary. In some embodiments, the capillary tip transfers sample into one or more channels, and provides increased patient comfort and safety.

Referring to FIGS. 20-22, in some embodiments, the collection device further includes an assembly feature (e.g., element 2060 or 2260) to accommodate assembly of sample collection medium (e.g., an FTA® paper) for providing a final collection device. The assembly feature couples the capillary to the cap operatively and firmly to provide a final collection device using an assembly method e.g., press-fit, glue, ultrasonic welding, rivet, or any other methods. In some embodiments, the assembly feature includes one or more pins, a flat plate, a secondary cap of any shapes, or any other configurations. In some embodiments, a sample collection medium, such as an FTA® paper, is assembled into the cap for collection of biological samples.

In some embodiments, the collection device includes a component or feature configured to provide surface treatment to separate the different components of blood (e.g., cellular, bacterial, or viral nucleic acids, proteins, antibodies, or polypeptides).

In some embodiments, the collection device includes a series of interior or exterior features (e.g., a ring or bubble blower, and/or urchin structures) configured to collect and/or hold fluids.

Referring to FIGS. 23 and 24, in some embodiments, the collection device in the present disclosure includes one or more mechanical devices, such as a suction device, a duck-bill valve, and/or other suitable devices for sample collection and/or retention of samples.

In some embodiments, the suction device can be operatively coupled to either or both ends of the cap to aid in collecting samples (e.g., blood or other fluids) by suctioning. For example, as shown in FIG. 23, the suction device 2360 is operatively attached to a first end (e.g., opening 128) of the cap 120, where the first end is away from the proximal end 2302 of the sample collection vehicle 2310. In another example, as shown in FIG. 24, the suction device 2360 is operatively attached to a second end of the cap 120, where the second end is closer to the proximal end 2302 of the sample collection vehicle 2310.

In some embodiments, the suction device can be operated by hand or by a machine (e.g., automated machine).

In some embodiments, the suction device is removable from the cap to keep the automation compatibility of the collection device. In some embodiments, the suction device is locked with the cap in use, and can be detached after samples are collected.

Optionally, the suction device includes at least one frangible connection (or breakpoint) for removing the suction device by applying an external force. In some embodiments, the frangible connection has a lower strength under shear, torsional, and/or compressive forces than the other portions of the suction device. In some embodiments, an external force includes a single direction bend, torsion, twisting, or any combinations thereof, at the same time or at different times. For example, a user could snap off, twist off, pull off, or otherwise cut the suction device at the frangible connection.

As shown in FIG. 24, the collection device can further include a duck-bill valve 2460 to aid in sample collection and/or retention of samples during transport. In some embodiments, the duck-bill valve 2460 is attached to the proximal end 2302 of the sample collection vehicle 2310. In some embodiments, the duck-bill valve 2460 is located in proximity to the proximal end 2302 of the sample collection vehicle 2310.

In some embodiments, the sample collection vehicle 2310 as shown in FIGS. 23 and 24 refer to a capillary or other sample collection structures as described herein.

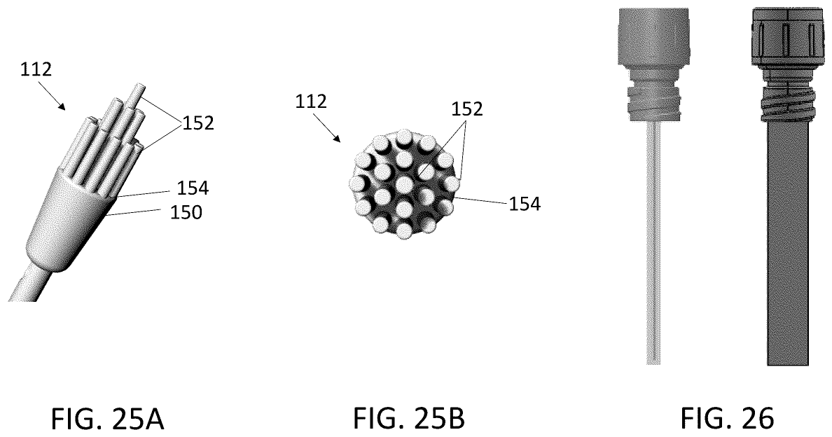

FIGS. 25A and 25B are schematic, detail views of an exemplary brush of a sample collection device, according to various embodiments. In some embodiments, as shown in FIGS. 25A and 25B, the sample collection device includes a brush head 112 that includes a support portion 150 and a plurality of sample collection bristles 152 attached to and extending from the support portion 150. In some embodiments, the sample collection bristles 152 extends substantially perpendicular from a surface 154 of the support portion 150 as shown in FIG. 25A. In some embodiments, the sample collection bristles 152 are configured to be a plurality of rods (FIG. 25B) or other forms of filaments. In some embodiments, the sample collection bristles 152 are cervical bristles that are configured to include a plurality of sampling sheets, such that the bristles 152 can be conveniently adhered to a sample collection area (e.g., the external orifice of the cervical canal and cells on the surface of the cervix).

In some embodiments, the brush head 112 can take any forms.

In some embodiments, the sample collection bristles 152 include a length of in a range of about 1 mm to about 10 mm. In some embodiments, the sample collection bristles 152 includes a length of about any of: 1 mm, 5 mm, or 10 mm. In some embodiments, the sample collection bristles 152 are configured to include different lengths. In some embodiments, the length of the sample collection bristles 152 taper from a maximum length at the bristle extending from the center of the surface 154 of the support portion 150 to a minimum length at the bristle extending from the outer area of the surface 154 of the support portion 150.

In some embodiments, the one or more sample collection bristles are spaced apart. In some embodiments, the one or more sample collection bristles include any of: 1, 2, 3, 4, 5, 6, 7, 8, 9, 10, 11, 12, 13, 14, 15, 16, 17, 18, 19, 20, 21, 22, 23, 24, 25, or more bristles.

In some embodiments, an sample collection bristle 152 includes a diameter or a width in a range of about 0.5 mm to about 5 mm, about 1 mm to about 4.5 mm, about 1.5 mm to about 4 mm, about 2 mm to about 3.5 mm, about 2.5 mm to about 3 mm. In some embodiments, the annular ring(s) 113 includes a thickness in a range of about 0.05 mm to about 3 mm, about 0.1 mm to about 2.5 mm, about 0.5 mm to about 2 mm, about 1 mm about 1.5 mm. In some embodiments, the sample collection bristles 152 are configured to include different diameters or widths.

In some embodiments, the collection devices disclosed herein include additional features such as frangible feature, self-locking feature, anti-rotation feature, and/or other features. Examples of these features are described in more details in U.S. Provisional Application No. 63/326,465 and applications claiming priority thereto, all of which are incorporated herein by reference.

### Materials for Manufacturing a Collection Device

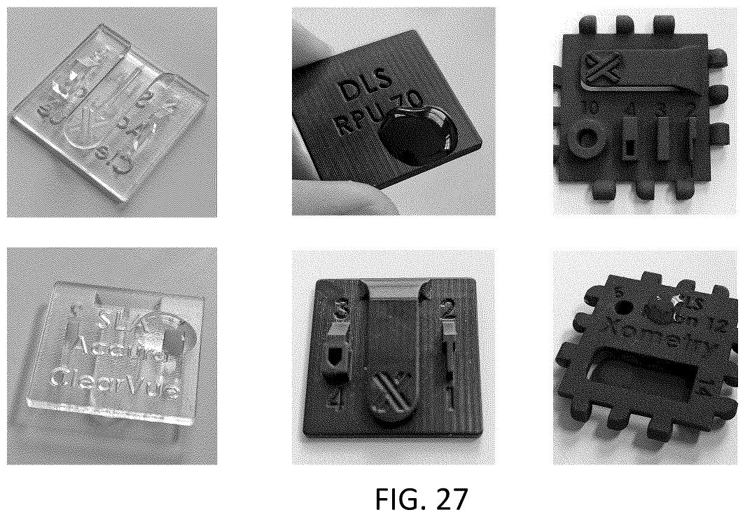

FIG. 27 are images of embodiments of materials used for manufacturing a collection device. In some embodiments, at least some of the materials used for manufacturing a collection device, as described herein, can be used for manufacturing a vial.

In some embodiments, one or more materials can be used to manufacture at least one component of a collection device (e.g., capillary, cap, or their components), according to various embodiments.

In some embodiments, the collection device material exhibits at least one of the following characteristics: (1) sufficiently rigid for collection of samples; (2) sufficiently flexible for safety of use; (3) collects adequate sample for subsequent tests; (4) withstands the rigors of sterilization/disinfection without structural weakening, or chemically interfering subsequent testing. (5) compatible with subsequent testing (e.g., PCR testing and/or nucleic acid extraction technologies, antigen testing, antibody-based testing).

In some embodiments, the collection device material is biodegradable. In some embodiments, the biodegradable collection device materials include a biobased plastic, polyhydroxyalkanoate (PHA), polylactic acid (PLA), starch blend, cellulose-based plastic, lignin-based polymer composite, a petroleum-based plastic, polyglycolic acid (PGA), polybutylene succinate (PBS), polycaprolactone (PCL), poly(vinyl alcohol) (PVA, PVOH), polybutylene adipate terephthalate (PBAT), polyethylene terephthalate glycol (PETG), polypropylene (PP), Pebax, gelatin, collagen, chitosan, starch, sucrose, soy based materials, or other natural-sugar based materials, materials incorporate enzymatically labile groups, and/or their derivatives or combinations.

In some embodiments, the collection device material is water-soluble. In some embodiments, e.g., after the contacting step, at least a portion of the water-soluble collection device (e.g., the soluble portion) is dissolved, e.g., with water or an aqueous solution. Such a dissolving step can permit faster release of the sample from the collection device for downstream applications. In some embodiments, the dissolving step can implement a colorimetric change or other diagnostic detection methods.

In some embodiments, the collection device material is a polymer. In some embodiments, the collection device material includes at least one of polypropylene, polycarbonate, thermoplastic elastomers (TPE), rubber, polyester fiber, acrylonitrile butadiene styrene (ABS), acrylic, polyetherimide, ionomer, acetal copolymer, polyurethane, polystyrene, nylon, or any combination thereof.

In some embodiments the collection device material includes polyvinyl alcohol or a derivative polymer such as polyvinyl acetals, polyvinyl butyral (PVB), or polyvinyl formal (PVF). In some embodiments, the collection device material comprises Kuraray MOWIFLEX™ C17 or C30 materials, which are PVA variants. In some embodiments, the collection device material comprises Kuraray POVAL™ materials.

In some embodiments, the collection device material is glass.

In some embodiments, the collection device material is plastic.

In some embodiments, the collection device material is a solid material (i.e., non-porous).

In some embodiments the collection device material is flocked/fibrous.

In some embodiments the collection device material is hydrophilic.

In some embodiments, the collection device material is a foam.

In some embodiments, the collection device material includes a paper (e.g., FTA paper) or pulp substrate.

In some embodiments, the collection device material is nonwoven.

In some embodiments, the collection device material includes bicomponent fibers.

In some embodiments, the collection device material includes cotton or other natural fibers.

In some embodiments, the collection device material includes synthetic fibers.

In some embodiments, the collection device material includes engineered films with hydrophilicity or other material properties.

In some embodiments, the collection device material is hydrophobic.

In some embodiments, the collection device material is a porous material.

In some embodiments, the collection device material is medical grade.

In some embodiments, the collection device material exhibits the following features: autoclave sterilizable; E-beam sterilizable; ethylene oxide sterilizable; no animal derived components; and radiation sterilizable.

In some embodiments, the collection device material can withstand surface treatments such as chemical treatments, surface modifications (e.g., plasma treatment), and/or mechanical treatments (e.g., surface texturing, cross-hatching, or high polish), or any other treatments.

In some embodiments, the collection device material has a flexural modulus (also referred to as bending modulus, which is the ratio of stress to strain in flexural deformation, or the tendency for a material to resist bending) of about 100 megapascals (MPa) to 5000 MPa.

### Manufacture of a Collection Device

In some embodiments, at least one component of the collection device (e.g., capillary, cap or their components as described herein) is manufactured using molding (e.g., injection molding, or overmolding), stamping, die cutting, thermal cutting, ultrasonic welding, extrusion, milling, lamination, or 3D printing. For example, at least one component of the collection device (e.g., capillary, cap or their components as described herein) is configured to be ejected from a mold.

In some embodiments, at least one component of the collection device (e.g., capillary, cap or their components as described herein) is manufactured so as to not result in abrasive or sharp features, in order to avoid damage to the subject and ensure a safe use of the collection device.

In some embodiments, at least one component of the collection device 100 is fabricated from at least one polymer (e.g, polypropylene, and/or other suitable polymer materials). In some embodiments, at least one component of the collection device is fabricated from glass.

In some embodiments, a method of manufacturing the at least one component of a collection device includes: 1) manufacturing a mold, e.g., according to the dimensions and/or features of the collection device; 2) injecting a mold with a liquid form of one or more collection device material(s); and/or 3) removing at least a portion of the collection device (e.g., the capillary) from the mold once solidified.

Referring to FIG. 26, in some embodiments, the method of manufacturing of the collection device includes injection molding. In particular embodiments, the method of manufacturing of the collection device includes a two-shot injection molding, where a first shot includes manufacturing cap and mechanical feature to hold the material manufactured in a second shot. In some embodiments, the second shot material can be determined to achieve desired sample collection performance. In some embodiments, examples of materials used in second shot are selected from hydrophilic polymers, foams, soluble collection mediums, or any other suitable materials.

In some embodiments, the collection device material(s) is liquefied, e.g., at a temperature of about 420° C.

In some embodiments, at least one component of the collection device may be molded separately.

In some embodiments, at least part of the collection device may be configured to be ejected from a mold. In some embodiments, at least part of the collection device may be configured (e.g., machined) to include a non-abrasive feature instead of abrasive or sharp features, in order to avoid damage to the subject and ensure a safe use of the collection device.

In some embodiments, the method of manufacturing of the collection device includes a demolding step such that the final product (e.g., collection device) is removed from the mold.

### Vial

In some embodiments, the threaded portion 122 of the cap 120 is configured to seal with and/or interlock with the opening of a vial. For example, after the sample is collected, the threaded portion (e.g., external or internal threads) of the collection device may be screwed to the threaded portion of the vial.

In some embodiments, the cap 120 and/or the vial may include an external and/or internal structure (e.g., O-ring or gasket) to aid in forming a substantially fluid-tight seal between the cap 120 and the vial and in stopping liquid from leaking from the vial.

In some embodiments, the maximum diameter of the cap 120 is greater than, or substantially the same as the diameter of the opening of the vial.

In some embodiments, the vial contains sample transport media. In other embodiments, the vial is dry before in use.

In some embodiments, the vial can be constructed from a transparent material.

In some embodiments, the vial includes a length (e.g., a distance between the opening of the vial and the bottom of the vial) that is substantially the same as or greater than the length of the shortened capillary of the collection device.

In some embodiments, the vial has an inner diameter that is greater than the maximum diameter of the capillary inserted in the vial.

In some embodiments, the vial includes a threaded portion at or in proximity to the opening of the vial. In some embodiments, the threaded portion of the vial includes at least 1, 2, 3, 4, or 5 threads (e.g., internal and/or external threads) to allow the cap 120 to be screw onto the vial. In some embodiments, the threads are continuous and/or discontinuous.

In some embodiments, the vial is compatible for use with an automated device. In some embodiments, the vial is compatible with the Society for Biomedical Sciences (SBS) 24-well format, the SBS 48-well format, the SBS 96-well format, or any combination thereof.

In some embodiments, the vial (e.g., vials in FIGS. 4-6) includes a volume from about 1 ml to about 10 ml. In some embodiments, the vial includes a volume from about 0.1 ml to about 20 ml. In some embodiments, the vial includes a volume from about 1 ml to about 8 ml. In some embodiments, the vial includes a volume from about 3 ml to about 6 ml. In preferred embodiments, the vial includes a volume from about 4 ml to about 5 ml. In preferred embodiments, the vial includes a volume of about 4 ml, 4.5 ml, or 5 ml.

In some embodiments, the vial includes a barcode at the outside surface of the vial.

In some embodiments, the vial includes an average width or diameter from about 1 mm to about 100 mm, from about 5 mm to about 100 mm, from about 10 mm to about 100 mm, from about 15 mm to about 95 mm, from about 20 mm to about 90 mm, from about 25 mm to about 85 mm, from about 30 mm to about 80 mm, from about 35 mm to about 75 mm, from about 40 mm to about 70 mm, from about 45 mm to about 65 mm, from about 50 mm to about 60 mm. In some embodiments, the vial includes an average width or diameter of about any of: 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm. In some embodiments, the vial includes an average width or diameter of at least about any of: 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm. In some embodiments, the vial includes an average width or diameter of at most about any of: 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm. In some embodiments, the vial includes any desired volume (e.g., a universal volume).

In some embodiments, the vial includes a height from about 10 mm to 100 mm, about 15 mm to about 95 mm, about 20 mm to about 90 mm, about 25 mm to about 85 mm, about 30 mm to about 80 mm, about 35 mm to about 75 mm, about 40 mm to about 70 mm, about 45 mm to about 65 mm, or about 50 mm to about 60 mm, about 50 mm to about 55. In some embodiments, the vial includes an average height of about any of: 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm. In some embodiments, the vial includes an average height of at least about any of: 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm. In some embodiments, the vial includes an average height of at most about any of: 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm. In some embodiments, the vial includes any desired height (e.g., a universal height).

In some embodiments, the vial is coupled to the cap. Such vial-cap assembly includes a height from about 10 mm to about 120 mm, about 20 mm to about 110 mm, about 30 mm to about 100 mm, about 40 mm to about 90 mm, about 50 mm to about 80 mm, about 60 mm to about 70 mm. In some embodiments, the vial-cap assembly includes a height of about any of: 10 mm, 20 mm, 30 mm, 40 mm, 50 mm, 60 mm, 70 mm, 80 mm, 90 mm, 100 mm, 110 mm, or 120 mm. In some embodiments, the vial-cap assembly includes a height of at least about any of: 10 mm, 20 mm, 30 mm, 40 mm, 50 mm, 60 mm, 70 mm, 80 mm, 90 mm, 100 mm, 110 mm, or 120 mm. In some embodiments, the vial-cap assembly includes a height of at most about any of: 10 mm, 20 mm, 30 mm, 40 mm, 50 mm, 60 mm, 70 mm, 80 mm, 90 mm, 100 mm, 110 mm, or 120 mm.

In some embodiments, the vial can include a cross-section of at least one shape of a polygonal shape, e.g., a triangle, a square, a quadrilateral, a trapezoid, a pentagon, a hexagon, a polygon with at least 7, 8, 9, 10, 11, 12, 13, 14, 15, 16, 17, 18, 19, 20 or more sides, a circle, a circle with one or more flat sides, an ellipse, or an ellipse with one or more flat sides. In some embodiments, at least one side of the cross-section includes a convex and/or concave curve. In some embodiments, the cross-section includes a rotationally symmetric shape or an asymmetric shape. In some embodiments, the cross-section includes the same shape, or at least two different shapes for the entirety of the component.

In some embodiments, the vial is automation compatible and can interface with an automation device (e.g., a decapper machine or handheld decapper).

In some embodiments, the decapper machine or handheld device is selected from any one of AltemisLab Advantage 96, Altecap Switch, IntelliXcap, Capit-All, Screw Cap CS700, Univo SR048, SafeCap, or Micronic, LVL, Hamilton Storage or Robotics, ThermoFisher, Azenta, RHINObot, or Micronics. In particular embodiments, the decapper machine or handheld device is manufactured or sold by Rhinostics or other companies.

In some embodiments, the decapper machine or handheld device is attached to and works with a liquid handler. In some embodiments, the liquid handle is selected from any one of Hamilton, Tecan, Open-trons, or any other manufacturers.

In some embodiments, the vial is selected from any one of Corning, Nest, AltemisLab, Altertube, Azenta Cryotube, Thermo-Nunc, Thermo-Matrix, Micronic, Ziath Cryzotraq, Globe CryoClear, LVL, Thermo-Nalgene, United Scientific, Cryovial, or Labforce (Thomas). In particular embodiments, the vial is manufactured by Rhinostics or other companies.

In some embodiments, the vial is compatible with a rack (e.g., SBS 24-well format, the SBS 48-well format, the SBS 96-well format, any desired size (e.g., a universal size) of well formats, and/or other suitable formats) as described in further detail below.

In some embodiments, an element (e.g., an elastomeric disk) may be used for sealing the cap and vial. In some embodiments, the element extends inward to the capillary for sealing and holding the components of the shortened collection device in place during decapping.

In some embodiments, the vial includes any desired volume (e.g., a universal volume). In some embodiments, the vial includes a volume from about 1 ml to about 10 ml. In some embodiments, the vial includes a volume from about 0.1 ml to about 20 ml. In some embodiments, the vial includes a volume from about 1 ml to about 8 ml. In some embodiments, the vial includes a volume from about 3 ml to about 6 ml. In preferred embodiments, the vial includes a volume from about 4 ml to about 5 ml. In preferred embodiments, the vial includes a volume of about 4 ml, 4.5 ml, or 5 ml.

In some embodiments, the vial includes a barcode at the outside surface of the vial.

In some embodiments, the vial includes an average width from about 10 mm to about 15 mm. In some embodiments, the vial includes an average width from about 11 mm to 14 mm. In preferred embodiments, the vial includes an average width from about 12 mm to 13 mm. In preferred embodiments, the vial includes an average width of about 12, 12.1, 12.2, 12.3, 12.4, 12.5, 12.6, 12.7, 12.8, 12.9 or 13.0 mm. In some embodiments, the vial includes an average width of about 0.5 mm.

In some embodiments, the vial includes a height from about 1 mm to 100 mm. In some embodiments, the vial includes a height from about 50 mm to 100 mm. In some embodiments, the vial includes a height from about 60 mm to 70 mm. In some embodiments, the vial includes a height from about 1 mm to 50 mm. In some embodiments, the vial includes a height from about 1 mm to 10 mm. In some embodiments, the vial includes a height of about 1, 2, 3, 4, 5, 6, 7, 8, 9, 10, 20, 30, 40, 50, 60, 70, 80, 90, or 100 mm.

In some embodiments, the vial is coupled to the cap. Such vial-cap assembly includes a height from about 1 mm to 120 mm. In some embodiments, the vial-cap assembly includes a height from about 50 mm to 120 mm. In some embodiments, the vial-cap assembly includes a height from about 50 mm to 90 mm. In some embodiments, the vial-cap assembly includes a height from about 1 mm to 50 mm. In some embodiments, the vial-cap assembly includes a height from about 1 mm to 10 mm. In some embodiments, the vial-cap assembly includes a height of about 1, 2, 3, 4, 5, 6, 7, 8, 9, 10, 20, 30, 40, 50, 60, 70, 80, 90, 100, 110, or 120 mm.

In some embodiments, the vial is automation compatible and can interface with an automation device (e.g., a decapper machine).

In some embodiments, the vial is compatible with a rack (e.g., SBS 24-well format, the SBS 48-well format, the SBS 96-well format, and/or other suitable formats) as described in further detail below.

in some embodiments, an element (e.g., an elastomeric disk) may be used for sealing the cap and vial.

In some embodiments, the element extends inward to the capillary for sealing and holding the components of the shortened collection device in place during decapping.

### Kits

In some embodiments, one or more kits (e.g., sample collection kit or test kit) may be used for collecting samples using the collection devices (e.g., collection device 100) as described herein.

In some embodiments, the kit includes 1, 2, 3, 4, 5, 6, 7, 8, 9, 10, 11, 12, 13, 14, 15, 16, 17, 18, 19, 20, 30, 40, 50, 60, 70, 80, 90, 100 or more collection devices and/or vials.

In some embodiments, the kit includes an effective amount of sample transport media. In some embodiments, the sample transport media is supplied in a lyophilized or dried form, a concentrated liquid form that can diluted or suspended in liquid prior to use with the collection device, or a liquid solution (e.g., an aqueous solution such as a sterile aqueous solution). In some embodiments, the sample transport media include boric acid, anticoagulants, DNA/RNA preservatives, other preservatives, and/or protective agents. Preferred formulations include those that are non-toxic to the samples (e.g., cells bacteria, viruses) and/or does not affect growth rate or viability. The sample transport media can be supplied in aliquots or in unit doses. In some embodiments, transport media preserves the sample components (e.g., cellular, bacterial, or viral nucleic acids, proteins, antibodies or polypeptides) nucleic acid between the time of sample collection and downstream applications.

In some embodiments, the sample transport media comprises a viral transport media (VTM). The constituents of suitable viral transport media are designed to provide an isotonic solution containing protective protein, antibiotics to control microbial contamination, and one or more buffers to control the pH. Isotonicity, however, is not an absolute requirement; some highly successful transport media contain hypertonic solutions of sucrose. Liquid transport media are used primarily for transporting collection devices or materials released into the medium from a collection device. Liquid media may be added to other specimens when inactivation of the viral agent is likely and when the resultant dilution is acceptable.

In some embodiments, the kit can optionally include one or more agents that permit the detection of cellular, bacterial, or viral nucleic acids or polypeptides in the sample (e.g., test strips).

In some embodiments, the kit optionally comprises informational material. The informational material can be descriptive, instructional, marketing or other material that relates to the methods described herein.

In some embodiments, the compositions in the kit can be provided in a watertight or gas tight container which in some embodiments is substantially free of other components of the kit. For example, the collection device can be supplied in at least one container (e.g., the vial), and the sample transport media can be supplied in a container having sufficient reagent for a predetermined number of samples, e.g., 1, 2, 3 or greater. It is preferred that the components described herein are substantially pure and/or sterile.

In one embodiment, the informational material can include information about production of any of the components (e.g., collection devices, vials, sample transport media), concentration, date of expiration, batch or production site information, and so forth. In one embodiment, the informational material relates to methods for collecting samples using the components of the kit.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time. In some embodiments, the kit can be provided with components of various sizes, e.g., capillaries and caps of varying dimensions (e.g., any of the lengths described herein), such that a user can select the appropriate capillary and/or cap for particular applications.

### Methods and Workflow of Using a Collection Device

In some embodiments, an example workflow using a collection device is described as below.

At step 1, a collection device including a capillary or other structure and/or a cap is obtained. In some embodiments, the collection device is included in a kit. In some embodiments, the collection device can be delivered/obtained with the capillary or other structure attached to the cap 120. In other embodiments, the collection device is delivered/obtained with the cap separate from the capillary, e.g., before in use.

At step 2, optionally, the cap is connected firmly to the capillary (e.g., from a distal end). The collection device can then be used to collect a sample (e.g., biological sample).

At step 3, the collection device, including the capillary and the cap, is sealed to the vial by screwing the threaded portion of the cap to the threaded portion of the vial. In some embodiments, the dimension and structure of the shortened collection device is configured to be fully and securely coupled to the opening of the vial.

At step 4, an automation device is used to remove the cap from the vial.

At step 5, the collection device, including the cap operatively coupled to the incomplete capillary, is detached from the vial after decapping.

In some embodiments, additional steps may include pricking or puncturing e.g., the dermis layer of the skin for the sample collection device to access e.g., the capillary beds that run through the subcutaneous layer of the skin.

In some embodiments, the workflow in the present disclosure further includes blood drying methods (e.g., for drying the blood in the cap or vial). In some embodiments, the workflow in the present disclosure further includes methods (e.g., surface treatment or other physical methods) to separate blood components.

In some embodiments, the collection can take place in a medical facility by a medical professional or self-collected by the patient. In other embodiments, the collection device can be sent to the patient in their home for self-collection and shipment back to the laboratory.

### Automation System and Process

In some embodiments, the collection device and/or the sample can be processed (e.g., after the collection device is deposited into a vial) using a manual process, a semi-automated process, or a fully automated (or automation) process. In some embodiments, an automation process includes using one or more automation devices. In some embodiments, the automation device includes a tube capper and decapper machine. In some embodiments, the automation device includes a liquid handling machine. In some embodiments, the automation device includes a shaker (e.g., an orbital shaker).

In general, the cap can have any structure that corresponds with any known or future developed automation device. For example, in some embodiments, the cap includes a hollow internal portion, an outer surface of the cap (e.g., top surface, circumferential surface, side surface), and/or one or more internal ribs, which enables the cap to interface with an automated device.

In some embodiments, an automation device can move, control, and/or manipulate at least a portion of the collection device after interfacing with the cap. In some embodiments, a portion of the automation device can extend into the hollow internal portion of the cap.

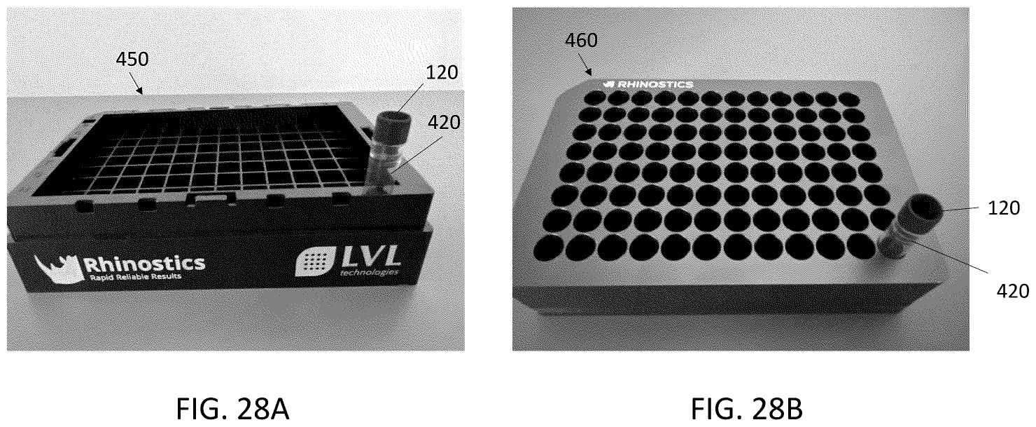

FIGS. 28A and 28B are views of embodiments of 96-well rack for storage of a collection device sealed to a vial 420, according to various embodiments. In some embodiments, the rack 450 includes one or more square wells (FIG. 28A). In some embodiments, the rack 460 includes one or more round wells (FIG. 28B).

In some embodiments, an automated method of processing a collection device includes using an automation system to perform at least one of the following steps: 1) receiving a collection device that has been contacted with a sample and deposited into a vial; 2) removing a cap from a vial; 3) removing at least a portion of a sample from the collection device and the vial (e.g., by removing a liquid within the vial and/or by removing the collection device); 4) transporting at least a portion of the sample to a testing location (as an alternative to steps 2) and 3) the sample can remain in the vial and a testing solution can be delivered into the vial); 5) testing at least a portion of the sample (e.g., to determine the presence of some substance) and capturing data resulting from the test; 6) further processing at least a portion of the sample.

In some embodiments, after receiving the collection device, a barcode (or a label) on the collection device and/or vial is detected using a barcode scanning machine.

In some embodiments, an automation system for processing the collection device includes one or more devices (e.g., a tube capper, a decapper machine, a liquid handling machine, and/or a shaker). In some embodiments, the one or more devices are automated machines (e.g., robots).

In some embodiments, the step of removing at least a portion of a sample from the collection device and the vial further includes: removing the collection device from the vial (e.g., using the tube capper and decapper machine), adding a solution to the vial (e.g., using a liquid handling machine), placing the collection device back into the vial (e.g., using the tube capper and decapper machine), shaking the solution in the vial using a shaker in order to remove at least a portion of the sample from the incomplete capillary of the collection device, removing the collection device from the vial and solution (e.g., using the tube capper and decapper machine, and/or removing a portion of the solution from the vial (e.g., using the liquid handling machine) for further processing (e.g., testing)

In some embodiments, the solution is saline.

In some embodiments, the further processing includes a diagnostic test. In some embodiments, the further processing includes nucleic acid (e.g., RNA or DNA) extraction, protein extraction, nucleic acid (e.g., RNA or DNA) amplification (e.g., PCR or isothermal amplification methods), and/or a detection assay (e.g., RT-qPCR). Non-limiting examples of isothermal amplification methods include: Recombinase Polymerase Amplification (RPA), Loop Mediated Isothermal Amplification (LAMP), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), and polymerase Spiral Reaction (PSR). In some embodiments, the tests can include antigen and/or protein-based tests. In some embodiments, the tests include analysis of immune or antibody status, vaccination status, presence or absence of protein biomarker, levels of protein biomarker, pathogen detection, and/or biological levels (e.g, A1c, hormone levels, or other biological levels). In some embodiments, the further processing includes mass spectrometry analysis or sequencing.

In some embodiments, the collection device described herein reduces the time of entire processes as described herein by in a range from about 1% to about 90%, about 1% to about 50%, about 5% to about 85%, about 10% to about 80%, about 15% to about 75%, about 20% to about 70%, about 25% to about 65%, about 30% to about 60%, about 35% to about 55%, or about 40% to about 50%, compared to a process using traditional methods (e.g., enzyme-linked immunoassay (ELISA), mass spectrometry, or other traditional methods to measure proteins and antibody levels).

In some embodiments, an automation method for processing a rack of one or more collection devices includes: 1) obtaining one or more vials, where the vial includes samples collected in a collection device and sealed to the vial using the cap (e.g., cap 120); 2) loading the vials (e.g., manually or by an automation process) to at least one rack (e.g., in a rack 450 or 460 in FIGS. 28A and 28B) for testing; 3) putting the rack of vials onto a robot for scanning (e.g., barcodes on the vials) e.g., in seconds; 4) passing the rack of vials to a decapping machine, where the automation device removes the caps (e.g., 96 caps) from the vials in the rack (e.g., in 30 seconds) and then moves the rack to a liquid handling robot (e.g., 30 seconds); 5) adding a saline solution (e.g., 100 uL in 10 seconds) using the liquid handling robot; 6) moving the rack back to the decapping robot e.g., from the liquid handling robot (e.g., 30 seconds); 7) replacing the caps (e.g., 30 seconds) of the vials; 8) moving the vials to e.g., an orbital shaker (e.g., 30 seconds), which shakes to move sample material into solution (e.g., 10 seconds); 9) moving the rack to the decapping robot (e.g., 30 seconds) and removing the caps (e.g., 30 seconds); 10) moving the rack back to the liquid handler (e.g., 30 second), which moves at least part of the sample into a microplate for e.g., downstream qPCR; 11) moving the rack back to the decapper robot, putting the caps back on; and 12) moving the rack to a storage location (e.g., 1.5 minutes). In some embodiments, the time for a portion or all the steps for processing a rack takes about 1, 2, 3, 4, 5, 6, 7, 8, 9, or 10 minutes. In some embodiments, the vials (or tubes) are decapped, followed by adding solution to the vials, and sending the vials into diagnostic instruments (e.g., instruments provided by Abbott m2000/Alinity; Roche, Hologic, Beckman Coulter) for further processing.

### Definitions

The term “a” or “an” refers to one or to more than one (i.e. at least one) of the grammatical objects of the article. By way of example “an analogue” means one analogue or more than one analogue.

The terms “about” or “substantially” that modify a condition or relationship characteristic of a feature or features of an embodiment of the invention, are to be understood to mean that the condition or characteristic is defined to within tolerances that are acceptable for operation of the embodiment for an application for which it is intended. For example, the terms “about,” “substantially,” and/or “close” with respect to a magnitude or a numerical value may imply to be within an inclusive range of −10% to +10% of the respective magnitude or value.

The term “overmolding” refers to a process wherein a single part is created using two or more different materials in combination. For example, the first material, sometimes referred to as the substrate, is partially or fully covered by subsequent materials (i.e., overmold materials) during the manufacturing process.

The term “subject,” “individual,” or “body,” refers to a human, an animal.

The term “complete capillary” or “closed capillary” refers to a capillary that includes a hollow interior that is entirely surrounded by the inner wall of the capillary. A cross-section of a complete capillary may include a closed shape whose line segments and/or curves form a closed region. In some embodiments, a “complete capillary” refers to a capillary that forms a fully enclosed lumen, e.g., the cross-sectional shape of the capillary forms an enclosed shape.

The term “incomplete capillary” or “open capillary” refers to a capillary including a cross-section of an open shape whose line segments and/or curves do not meet one or more endpoints of one side. In some embodiments, an “incomplete capillary” or “open capillary” refers to a capillary that forms a non-enclosed lumen, e.g., the cross-sectional shape of the capillary forms a non-enclosed shape.

The term “diameter” refers to the distance of a straight line passing through the axial center of a circular cross section. A diameter as described herein can include any one of a maximum, minimum, average, or medium diameter.

The term “automated” or “automation” refers to a device, system, or method that can be achieved using automatic means that are independent of human operators or supervision.

The term “automation compatible” refers to having features adapted to interact with any equipment that facilitates performance of a test or assay (e.g., decapping machine, handheld decapper equipment, liquid handlers, laboratory robotics, tube cappers, transport robotics, shaking apparatus, liquid transport mechanism, etc.).

The term “sample collection device” or “collection device” refers to a device that can be used to collect biological samples. The term “sample collection structure,” “collection structure,” or “sample collection vehicle” refers to a structure or element that can be used to collect biological samples. The biological samples can include biological fluids (e.g., blood) at any biological location (e.g., finger, arms, legs, or other suitable regions) of a subject (e.g., a human, an animal, etc.).

The term “channel” refers to a portion of a capillary where the fluids are collected, stored, conveyed through, or transported. In some embodiments, the term “channel” refers to the “cavities” (e.g., as shown in FIGS. 8-11). In some embodiments, a collection device can have 1, 2, 3, 4, 5, 6, or more channels.

Each numerical value presented herein is contemplated to represent a minimum value or a maximum value in a range for a corresponding parameter. Accordingly, when added to the claims, the numerical value provides express support for claiming the range, which may lie above or below the numerical value, in accordance with the teachings herein. Every value between the minimum value and the maximum value within each numerical range presented herein (including in the figures), is contemplated and expressly supported herein, subject to the number of significant digits expressed in each particular range. Absent express inclusion in the claims, each numerical value presented herein is not to be considered limiting in any regard.

Having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive. The terms and expressions employed herein are used as terms and expressions of description and not of limitation and there is no intention, in the use of such terms and expressions, of excluding any equivalents of the features shown and described or portions thereof. The structural features and functions of the various embodiments may be arranged in various combinations and permutations, and all are considered to be within the scope of the disclosed invention. Unless otherwise necessitated, recited steps in the various methods may be performed in any order and certain steps may be performed substantially simultaneously.

1. A collection device, comprising:
a capillary for collecting samples from a biological location of a body; and
a cap configured to interface with an automation device.

2. The collection device of claim 1, wherein the capillary comprises a cross-section of one or a plurality of U-shapes, V-shapes, L-shapes, double L shapes, curved shapes, polygonal shapes.

3. The collection device of claim 1, wherein the capillary comprises one or more sides, planes, overhangs, or combinations thereof.

4. The collection device of claim 1, wherein the capillary comprises a three-sided structure.

5. The collection device of claim 1, wherein the capillary comprises a parallel structure.

6. The collection device of claim 1, wherein the capillary is adapted to collect biological fluids samples.

7. The collection device of claim 1, wherein the capillary is a complete capillary.

8. The collection device of claim 1, wherein the capillary is an incomplete capillary.

9. The collection device of claim 1, wherein the cap is adapted to be secured to a vial prior to interfacing with the automation device.

10. The collection device of claim 1, wherein the cap comprises a threaded portion.

11. The collection device of claim 10, wherein the threaded portion is configured to interface with a threaded portion of the vial for sealing the vial.

12. The collection device of claim 1, wherein the automation device comprises at least one of a decapper machine and a handheld device.

13. The collection device of claim 1, further comprising one or more channels configured to improve sample travelling through the one or more channels.

14. The collection device of claim 1, further comprising one or more indicators at or near a distal end of the capillary and attached to the surface of the capillary, wherein the one or more indicators are configured to provide a visual signal indicating that the sample collected in the capillary reaches an indicator line.

15. The collection device of claim 1, further comprising a capillary tip operatively coupled to a proximal end of the capillary, wherein the capillary tip is configured to transfer the samples into one or more channels.

16. The collection device of claim 1, further comprising an assembly feature configured to accommodate assembly of a sample collection medium into the cap for collection of the samples.

17. The collection device of claim 16, wherein the sample collection medium comprises an FTA® paper.

18. The collection device of claim 16, wherein the assembly feature couples the capillary to the cap operatively and firmly to provide a final collection device using any one of press-fit, glue, ultrasonic welding, rivet, or combinations thereof.

19. The collection device of claim 1, wherein the collection device is configured to separate the different components of blood.

20. The collection device of claim 1, wherein the collection device comprises a suction device operatively coupled to either or both ends of the cap to aid in collecting the samples by suctioning.

21. The collection device of claim 1, wherein the suction device is operatable by an automated machine.

22. A sample collection device for collecting samples from a biological location of a body, comprising:
a support portion; and
a brush head extending substantially perpendicular from a surface of the support portion,
wherein the brush head comprises a plurality of sample collection bristles configured to include a plurality of sampling sheets, such that the bristles are conveniently adhered to a sample collection area of the biological location of the body.
